SEQUENCE LISTING (5 sequences)

SEQ ID NO: 1 - DNA, 1507 bp, Dictyoglomus thermophilum
  CDS (244)..(1302)
aagcttgaggaattgttgttgggtgatttgaagaaagaaggttatgatgtattttaccaatatgtctatattaaaaggaattttagatttgcggaaagaataaagagcctaatgaaagattacaaatctatatttgtggtaataggtgcaggtcatttaataggtgaagaaaacgtggtaaaattattacaaaattaggggttaaagtaagtaaaattttaatggaaaggggatgatggtaaaatgattaaccaaaggttttctatccttgtgcttcttctaattttgctcactttctcattaggatttttaaaggaggaggctaaaggtatggagattccatcactgaaagaagtttataaagattactttactattggagcggcagttagtcacttaaatatttatcattatgagaatcttttgaagaagcattttaatagtttaactcctgaaaatcaaatgaaatgggaagttattcatcctaaaccatatgtttatgattttggacctgccgatgagatcgtagattttgcaatgaagaatggtatgaaggtaagagggcatactttggtttggcataatcagactccaggatgggtttatgcaggtacaaaggatgaaattcttgcaagattaaaggaacacattaaagaggtagtaggacattataaaggtaaagtttatgcttgggatgtggttaacgaggccttatcggataatcctaatgaatttttgagaagagctccttggtatgatatttgtggtgaagaagttattgaaaaggcatttatttgggctcatgaggtagatcctgatgctaaattattttataacgattacaatttagaagatcctataaagagagaaaaggcttataaattagttaagaagctcaaggataaaggagttcctattcatggtattggaatacagggacattggacattagcatggccaactccaaagatgcttgaggattccataaagagatttgcagagcttggagttgaagtacaagttacagagtttgatatttctatttactacgatagaaatgaaaataataattttaaggttccgcctgaagataggcttgaaagacaagctcagctttataaggaggcttttgaaatcttaagaaaatacaaaggaatagttactggtgttactttctggggtgtagcagatgactatacttggctctatttctggcctgttagaggaagagaagattatccattactctttgataagaatcacaatcctaagaaggctttctgggagattgtaaagttttaggttgtataggaggtttaaatgagaaaattattaaggtttttgattttttctctattagttttaggactttctttcaatattattttagctgagactgagaaatatgtggataagaatttaggtttttctattgtgcctcctgaagggtgggaggtaaaggatggaaagccttataacttggctgtaatttttgttggccctgcag

SEQ ID NO: 2 - protein, 352 aa, Dictyoglomus thermophilum
MINQRFSILVLLLILLTFSLGFLKEEAKGMEIPSLKEVYKDYFTIGAAVSHLNIYHYENLLKKHFNSLTPENQMKWEVIHPKPYVYDFGPADEIVDFAMKNGMKVRGHTLVWHNQTPGWVYAGTKDEILARLKEHIKEVVGHYKGKVYAWDVVNEALSDNPNEFLRRAPWYDICGEEVIEKAFIWAHEVDPDAKLFYNDYNLEDPIKREKAYKLVKKLKDKGVPIHGIGIQGHWTLAWPTPKMLEDSIKRFAELGVEVQVTEFDISIYYDRNENNNFKVPPEDRLERQAQLYKEAFEILRKYKGIVTGVTFWGVADDYTWLYFWPVRGREDYPLLFDKNHNPKKAFWEIVKF

SEQ ID NO: 3 - DNA, 999 bp, Artificial
  Coding sequence for D. thermophilum xylanase without targeting sequence
atgtttctgaaagaagaagccaaaggtatggaaattccgagcctgaaagaagtgtataaagattactttaccatcggtgcagcagttagccatctgaatatttatcattatgagaacctgctgaaaaaacattttaatagcctgacaccggaaaatcagatgaaatgggaagtgattcatccgaaaccgtatgtgtatgattttggtccggcagatgaaattgttgattttgccatgaaaaatggcatgaaagttcgtggtcataccctggtttggcataatcagacaccgggttgggtttatgcaggcaccaaagatgaaattctggcacgcctgaaagaacatattaaagaagtggtgggccattataaaggtaaagtgtatgcctgggatgttgttaatgaagccctgagcgataatccgaatgaatttctgcgtcgtgcaccgtggtatgatatttgtggcgaagaagtgattgaaaaagcctttatttgggcacatgaagttgatccggatgccaaactgttttataatgattataatctggaagatccgattaaacgcgaaaaagcctataaactggtgaaaaaactgaaagataaaggcgttccgattcatggtattggtattcagggtcattggaccctggcatggccgaccccgaaaatgctggaagatagcattaaacgttttgccgaactgggtgttgaagttcaggtgaccgagttcgacatcagcatctactatgatcgcaatgaaaacaataactttaaagtgcctccggaagatcgtctggaacgtcaggcacagctgtataaagaagcctttgaaattctgcgcaaatacaaaggcatcgtgaccggcgtgaccttttggggtgttgcagatgattatacctggctgtatttttggccggttcgtggtcgtgaagattatccgctgctgtttgataaaaatcataatccgaaaaaagccttttgggaaattgtgaaattttaa

SEQ ID NO: 4 - protein, 332 aa, Artificial
  Synthetic Construct
MFLKEEAKGMEIPSLKEVYKDYFTIGAAVSHLNIYHYENLLKKHFNSLTPENQMKWEVIHPKPYVYDFGPADEIVDFAMKNGMKVRGHTLVWHNQTPGWVYAGTKDEILARLKEHIKEVVGHYKGKVYAWDVVNEALSDNPNEFLRRAPWYDICGEEVIEKAFIWAHEVDPDAKLFYNDYNLEDPIKREKAYKLVKKLKDKGVPIHGIGIQGHWTLAWPTPKMLEDSIKRFAELGVEVQVTEFDISIYYDRNENNNFKVPPEDRLERQAQLYKEAFEILRKYKGIVTGVTFWGVADDYTWLYFWPVRGREDYPLLFDKNHNPKKAFWEIVKF

SEQ ID NO: 5 - DNA, 999 bp, Artificial
  Coding sequence for D. thermophilum xylanase without targeting sequence
atgtttttaaaggaggaggctaaaggtatggagattccatcactgaaagaagtttataaagattactttactattggagcggcagttagtcacttaaatatttatcattatgagaatcttttgaagaagcattttaatagtttaactcctgaaaatcaaatgaaatgggaagttattcatcctaaaccatatgtttatgattttggacctgccgatgagatcgtagattttgcaatgaagaatggtatgaaggtaagagggcatactttggtttggcataatcagactccaggatgggtttatgcaggtacaaaggatgaaattcttgcaagattaaaggaacacattaaagaggtagtaggacattataaaggtaaagtttatgcttgggatgtggttaacgaggccttatcggataatcctaatgaatttttgagaagagctccttggtatgatatttgtggtgaagaagttattgaaaaggcatttatttgggctcatgaggtagatcctgatgctaaattattttataacgattacaatttagaagatcctataaagagagaaaaggcttataaattagttaagaagctcaaggataaaggagttcctattcatggtattggaatacagggacattggacattagcatggccaactccaaagatgcttgaggattccataaagagatttgcagagcttggagttgaagtacaagttacagagtttgatatttctatttactacgatagaaatgaaaataataattttaaggttccgcctgaagataggcttgaaagacaagctcagctttataaggaggcttttgaaatcttaagaaaatacaaaggaatagttactggtgttactttctggggtgtagcagatgactatacttggctctatttctggcctgttagaggaagagaagattatccattactctttgataagaatcacaatcctaagaaggctttctgggagattgtaaagttttag

(19) United States
(12) Patent Application Publication
(10) Pub. No.: US 2013/0029382 A1
(43) Pub. Date: Jan. 31, 2013

(54) COMPOSITIONS AND METHODS RELATING TO DUAL ACTIVITY ENZYMES HAVING XYLANASE AND CELLULASE ACTIVITY

(71) Applicants: John C. Steffens, Chapel Hill, NC (US); Paul Oeller, Durham, NC (US); Laurent Fourage, Suriname (FR)

(72) Inventors: John C. Steffens, Chapel Hill, NC (US); Paul Oeller, Durham, NC (US); Laurent Fourage, Suriname (FR)

(73) Assignee: SYNGENTA PARTICIPATIONS AG, Basel (CH)

(21) Appl. No.: 13/574,786

(22) PCT Filed: Jan. 21, 2011
(86) PCT No.: PCT/US11/22068

§ 371 Date: Oct. 1, 2012

Related U.S. Application Data
(60) Provisional application No. 61/298,020, filed on Jan. 25, 2010.

(51) Int. Cl. C12P 19/14; C12P 19/20; C12P 7/14; C12P 19/24

(52) U.S. Cl. 435/94; 435/99; 435/96; 435/162

(57) ABSTRACT

Compositions and methods are provided for treating lignocellulosic material with a dual activity enzyme having xylanase and cellulase activity. The enzyme is stable and active at increased pH and increased temperatures. The present invention therefore provides methods for hydrolyzing lignocellulosic material, especially cellulose and hemicellulose, which are major components of the cell wall of non-woody and woody plants. The methods for hydrolyzing cellulose and hemicellulose can be used on any plant, wood or wood product, wood waste, paper pulp, paper product or paper waste or byproduct.

### STATEMENT OF PRIORITY

This application claims the benefit, under 35 U.S.C. §119 (e), of U.S. Provisional Application No. 61/298,020; filed Jan. 25, 2010, the entire contents of which is incorporated by reference herein.

### REFERENCE TO SEQUENCE LISTING SUBMITTED ELECTRONICALLY

An official copy of a Sequence Listing submitted electronically via EFS-Web as an ASCII-formatted Sequence Listing with a file named “9848-8WO_ST25.txt,” created on Dec. 15, 2010, having a size of 18,891 bytes and filed concurrently with the Specification is a part hereof and is herein incorporated by reference as if set forth in its entirety.

### FIELD OF THE INVENTION

The present invention relates generally to methods of using enzymes having xylanase and cellulase activity to process lignocellulosic materials.

### BACKGROUND OF THE INVENTION

Hemicelluloses are a group of plant-derived heteropolysaccharides, which are associated with cellulose and lignin. The most common hemicelluloses are xylan, glucuronoxylan, arabinoxylan, glucomannan and xyloglucan.

Xylanases catalyze endohydrolysis of the 1,4-β backbone of xylan, a heteropolymer of xylose. Xylanases are naturally produced by organisms such as algae, bacteria, fungi, gastropods and protozoa, which can use xylose as a carbon source for cellular metabolism. See, Prade (1995) Biotech. Genet. Eng. Rev. 13:100-131.

Commercial applications of xylanases in the feed, food/beverage and technical industries (e.g., biomass applications) vary. In the feed industry, xylanases are used in monogastric and ruminant feeds to increase digestibility and nutritive value of poorly digestible feeds (e.g., barley, silage and wheat). In the food/beverage industry, xylanases are used in fruit and vegetable processing to make nectars, purees and juices, in brewing and winemaking to hydrolyze mucilaginous substances in grains for fermentation, and in baking to improve dough quality (e.g., elasticity and strength). In the technical industry, xylanases are used in papermaking to reduce chlorine consumption and toxic discharge during bleaching of wood pulp, in textile processing to reduce or replace chemical retting, and in bioremediation/bioconversion to treat/recycle wastes and to produce biofuels and fine chemicals.

In many of these applications, xylanases are used in connection with various other lignocellulolytic enzymes such as cellulases, hemicellulases, ligninases, pectinases and proteases. Lignocellulolytic enzymes therefore have significant potential applications in the feed, food/beverage and technical industries. The present invention overcomes previous shortcomings in the art by providing new compositions and methods for efficiently processing lignocellulosic materials are needed.

### SUMMARY OF THE INVENTION

Compositions and methods are provided for processing lignocellulosic materials. The compositions include at least one dual activity enzyme wherein the enzyme has xylanase and cellulase activity and wherein the enzyme is active at a high temperature and a high pH. Dual activity enzymes include xylanases, particularly family 10 xylanases, that are thermotolerant and alkaliphilic. The dual activity enzymes exhibit both xylanase and cellulase activity. Because of the alkaliphilic nature of the enzymes, they can be prepared as compositions or formulations at a high pH, typically pH 8.5 or higher. Further, the enzymes retain activity at high temperatures. The compositions can also include additional enzymes for processing lignocellulosic materials such as cellulases, hemicellulases, ligninases, pectinases and proteases.

The dual activity enzymes of the invention can be used to transform an organism of interest. Such organisms include bacteria, fungi and plants. Thus, for example, plants, plant parts and plant cells that have been modified to express at least one of the dual activity enzymes of the invention are disclosed.

The methods of the present invention involve the use of the dual activity enzymes and/or the compositions described herein to hydrolyze xylans and cellulose in lignocellulosic material in various feed, food/beverage and technical applications. The dual activity enzymes are useful for the bioconversion of lignocellulosic materials into simpler polysaccharides or even monosaccharides. These monosaccharides or polysaccharides can be used, for example, for the production of feed or food/beverages such as a cereal-based animal feed, a wort or beer, a milk or milk product, a fruit or vegetable product; for the production of fine chemicals such as butanol, ethanol, methanol and/or propanol; or for the production of fuels including biofuels such as bioethanol, bioethers, biodiesel and syngas. The dual activity enzymes are useful in the bioconversion of lignocellulosic materials because they are thermostable and can be used at a high pH.

Further aspects of the present invention provide methods wherein the dual activity enzyme is provided in the form of a plant, plant part or plant cell transformed with and expressing the dual activity enzyme.

Thus, in one embodiment of the present invention a method of processing lignocellulosic material is provided, the method comprising contacting the lignocellulosic material with an alkaline composition at a pH of at least about 10 and an enzyme comprising the amino acid sequence of SEQ ID NO:4, whereby the lignocellulosic material is processed. In some embodiments of the present invention, a method is provided wherein the lignocellulosic material is provided by a plant, plant part and/or plant cell transformed with a nucleotide sequence encoding the amino acid sequence of SEQ ID NO:4, thereby providing the enzyme comprising the amino acid sequence of SEQ ID NO:4 in the lignocellulosic material. Thus, in some embodiments, a method of processing lignocellulosic material is provided, the method comprising contacting the lignocellulosic material with an alkaline composition having a pH of at least about 10, wherein the lignocellulosic material comprises a plant, plant part or plant cell transformed with an enzyme having the amino acid sequence of SEQ ID NO:4, whereby the lignocellulosic material is processed.

In some embodiments, the lignocellulosic material is obtained from a plant, plant part or plant cell transformed with an active variant or active fragment of the amino acid sequence of SEQ ID NO:4. In some embodiments, the lignocellulosic material further comprises lignocellulosic material from a plant, plant part or plant cell not transformed with an enzyme having the amino acid sequence of SEQ ID NO:4.

In other embodiments, the methods of the invention further comprise contacting the lignocellulosic material with at least one additional enzyme is selected from the group consisting of a cellulase, hemicellulase, ligninase, pectinase, protease, and any combination thereof. In some particular embodiments, the cellulase is provided by the enzyme having the amino acid sequence of SEQ ID NO:4. In other embodiments, the cellulase is selected from the group consisting of a mannan endo-1,4-β-mannosidase, 1,3-β-D-glucan glucanohydrolase, 1,3-β-glucan glucohydrolase, 1,3-1,4-β-D-glucan glucanohydrolase, 1,6-β-D-glucan glucanohydrolase, and any combination thereof.

In some embodiments, the hemicellulase is an α-L-arabinofuranosidase, α-glucuronidase, acetyl mannan esterase, acetyl xylan esterase, α-galactosidase, β-glucosidase, β-1,4-xylosidase, endo-galactanase, endo-β-1,4-mannanase, endo-α-1,5-arabinanase, exo-β-1,4-mannosidase, exo-β-1,4-xylosidase, feruloyl esterase, ferulic acid esterase, p-cumaric acid esterase, glucuronoxylan xylanohydrolase, xyloglucan endotransglycosylase, or any combination thereof.

In other embodiments, the ligninase is a diarylpropane peroxidase, glucose oxidase, glyoxal oxidase, lignin peroxidase, manganese peroxidase, methanol oxidase, methanol oxidoreductase, phenol oxidase, phenol peroxidase, veratryl alcohol oxidase, or any combination thereof. In still other embodiments, the pectinase is a pectolyase, pectozyme, polygalacturonase, or any combination thereof. In some aspects of the present invention, the protease is an asclepain, bromelain, caricain, chymopapain, collagenase, glycyl endopeptidase, pepsin, pronase, subtilisin, thermolysin, or any combination thereof.

Additional embodiments of the present invention provide contacting the lignocellulosic material with at least one enzyme selected from the group consisting of an amylase, catalase, cutinase, glucanase, glucoamylase, glucose isomerase, lipase, phytase, pullulanase, xylose isomerase, and any combination thereof.

In other embodiments, the methods of the present invention further comprises contacting (e.g., fermenting) the lignocellulosic material with an ethanologenic bacteria or yeast or a combination thereof (at a pH of about 6 to about 8).

In still other embodiments, the present invention provides methods of pretreating the lignocellulosic material with a biological pretreatment, a chemical pretreatment, a physical pretreatment, or any combination thereof.

These and other aspects of the invention will be set forth in more detail in the description of the invention that follows.

### DETAILED DESCRIPTION OF THE INVENTION

The work described herein is the first to show that xylanases isolated from the thermophilic microorganism Diciyoglomus spp. can have dual activity. That is, the xylanases described herein not only hydrolyze xylan, but also hydrolyze cellulose. These enzymes retain significant xylanase and cellulase activity at a high pH and high temperature. Xylan is a major component of hemicellulose, and together with cellulose and lignin is collectively known as lignocellulose, making up the major constituents of non-woody and woody plants.

Various embodiments of the invention are described herein. Any of the features of the various embodiments of the invention described herein can be combined, creating additional embodiments which are intended to be within the scope of the invention.

As used herein, “lignocellulose” or “lignocellulosic” plant material means non-woody and woody plant material such as crop, plant and tree biomass comprising polysaccharide polymers (e.g., cellulose, hemicellulose and lignin). Lignocellulosic material also can include crop, plant or tree waste products including agricultural residues such as corn stover and sugarcane bagasse, dedicated energy crop residues, wood residues such as sawmill and paper mill discards, and municipal paper waste. In some embodiments, lignocellulosic plant material includes pulp from processing industries such as paper making.

The compositions and methods described herein therefore can be used to process lignocellulosic material to many useful organic chemicals, fuels and products. For example, some commodity and specialty chemicals that can be produced from lignocellulosic material include, but are not limited to, acetone, acetate, butanediol, cis-muconic acid, ethanol, ethylene glycol, furfural, glycerol, glycine, lysine, organic acids (e.g., lactic acid), 1,3-propanediol, polyhydroxyalkanoates, and xylose. Likewise, animal feed and various food/beverages can be produced from lignocellulosic material. See generally, Lynd et al. (1999) Biotechnol. Prog. 15:777-793; Philippidis, “Cellulose bioconversion technology” pp 179-212 In: Handbook on Bioethanol: Production and Utilization, ed. Wyman (Taylor & Francis 1996); and Ryu & Mandels (1980) Enz. Microb. Technol. 2:91-102. Potential co-production benefits extend beyond the synthesis of multiple organic products from fermentable carbohydrate in lignocellulosic material, as lignin-rich residues remaining after processing can be converted to lignin-derived chemicals or can be used for power production.

Compositions

Enzyme Compositions

Compositions of the invention include at least one dual activity enzyme or active variant or fragment thereof where the compositions are formulated at a high pH. The dual activity enzyme has xylanase and cellulase activity. By “high pH” is intended a pH of at least about pH 8.5, including a pH of about 9.0, about 9.5, about 10.0, about 10.5, about 10.0, about 11.5, about 12.0, about 12.5 or higher. Other components can be included within the compositions including other enzymes.

By “dual activity” enzyme is intended an enzyme having xylanase and cellulase activity. The xylanase activity of the enzymes of the present invention are active at high temperatures and high pH, while the cellulase activity is retained at these extremes of pH and temperature but becomes active upon the lowering of the pH to about 6 to about 8. The dual activity enzymes of the invention are classified as family 10 xylanases, which are both thermotolerant and alkaliphilic.

Xylanases are a large family of hydrolytic enzymes. As used herein, “xylanase” or “xylanases” means an enzyme capable of at least hydrolyzing xylan to xylobiose and xylotriose. Because of the heterogeneity and complexity of xylan, xylanases are quite diverse in their folding patterns (i.e., primary sequence variations), hydrolytic activities (i.e., yields, rates and products), mechanisms of action, substrate specificities and physicochemical characteristics. See, Methods in Plant Biochemistry and Molecular Biology (Dashek ed., CRC Press 1997); and Collins et al. (2005) FEMS Microbiol. Rev. 29:3-23. The official name for xylanase is endo-1,4-β-xylanase; however, it also is known in the art as endoxylanase, 1,4-β-D-xylan-xylanohydrolase, endo-1,4-β-D-xylanase, β-1,4-xylanase and β-xylanase.

Xylanases are classified with other glycosidases by primary structure comparisons of the catalytic domains and grouped in families of related sequences. See, Henrissat et al. (1989) Gene 81:83-95; and Henrissat et al. (2001) Methods Enzymol. 330:183-201. Within this classification system, xylanases are typically confined to glycosidase hydrolase families 10 and 11. Because of commercial demands for xylanases in feed, food/beverage and technical industries, a number of “extremophilic” xylanases have been isolated, especially xylanases from acidophilic, alkaliphilic and thermophilic bacteria.

Of interest herein are high-temperature (i.e., thermostable) and high-pH (i.e., alkaliphilic) tolerant xylanases isolated from microorganisms (e.g., bacteria) that exhibit both xylanase and cellulase activity. In one embodiment, the xylanase can be from a Dictyoglomus sp. such as Dictyoglomus thermophilum Rt46B.1. See, e.g., Gibbs et al. (1995) Appl. Environ. Microbiol. 61:4403-4408.

Accordingly, in some embodiments of the present invention, the amino acid sequences for the xylanase of the present invention are set forth in SEQ ID NOS:2 and 4. SEQ ID NO:4 differs from SEQ ID NO:2 by the deletion of the targeting sequence (amino acids 2-21) of SEQ ID NO:2. In other embodiments, the nucleotide sequence of the xylanase of the present invention comprises any nucleotide sequence that encodes the amino acid sequence of SEQ ID NO:2 and/or SEQ ID NO:4. Thus, in some particular embodiments, the nucleotide sequences for xylanases of the present invention are set forth in SEQ ID NO:1 and SEQ ID NO:3. In further embodiments, the nucleotide sequence of the present invention can be SEQ ID NO:1, wherein nucleotides 4-63 of SEQ ID NO:1 are deleted (i.e., SEQ ID NO:5). In other embodiments of the invention, the coding sequence for the enzyme comprises nucleotides 244 to 1302 of SEQ ID NO:1. See also, GenBank® Accession Nos. L39866 and AAA96979.

As discussed above, these enzymes show optimal xylanase activity at high temperatures and are thermostable. The half-life of the enzymes at 85° C. can be about 24 hours and 6.5 hours at pH 6.8 and pH 9.3, respectively. These enzymes are also alkaliphilic showing substantial xylanase activity across a broad pH range.

As indicated, the dual activity enzymes also have cellulase activity. “Cellulase” or “cellulases” means an enzyme capable of hydrolyzing cellulose to β-glucose. The cellulase activity of the dual activity enzymes is optimal at about 95° C. The cellulase activity also is thermostable, remaining even after 5 hours of incubation at about 95° C. The enzymes also show substantial cellulase activity across a broad pH range (e.g, pH6 to pH8.5), with the optimal activity at about pH 6.0.

The dual activity enzymes are thermotolerant and exhibit xylanase and cellulase activity at a temperature of at least about 60° C. By “thermotolerant” is meant that the enzyme retains at least about 70% activity at about 60° C. for 30 minutes, at least about 65% activity at about 70° C. for 30 minutes, at least about 60% activity at about 80° C. for 30 minutes. The enzymes retain xylanase and cellulase activity at temperatures up to about 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., or more.

As used herein, “alkaliphilic” means that the enzyme retains about 60% to about 90% of its xylanase activity at pH 8.5, retains at least about 65% activity at pH 9.0, retains at least about 60% activity at pH 10.0. In some embodiments, the dual activity enzyme retains activity at pH 10 or higher. In other embodiments, the enzyme retains activity at pH 12.0 or higher.

As used herein, “about” means within a statistically meaningful range of a value such as a stated concentration, time frame, weight (e.g., a percentage change (reduction or increase in weight)), volume, temperature or pH. Such a range can be within an order of magnitude, typically within 20%, more typically still within 10%, and even more typically within 5% of a given value or range. The allowable variation encompassed by “about” will depend upon the particular system under study, and can be readily appreciated by one of skill in the art.

As used herein, “a,” “an” or “the” can mean one or more than one. For example, “a” cell can mean a single cell or a multiplicity of cells.

As used herein, “and/or” refers to and encompasses any and all possible combinations of one or more of the associated listed items, as well as the lack of combinations when interpreted in the alternative (“or”).

As used herein, the transitional phrase “consisting essentially of means that the scope of a claim is to be interpreted to encompass the specified materials or steps recited in the claim” and those that do not materially affect the basic and novel characteristic(s)” of the claimed invention. Thus, the term “consisting essentially of” when used in a claim of this invention is not intended to be interpreted to be equivalent to “comprising.”

As used herein “to process,” “processed,” “processing” (and grammatical equivalents thereof) refers to the manipulation or modification of the lignocellulosic material and includes any mechanical, chemical or biological method for modifying the material. Processing includes incubating or holding plant material in a container for any period of time. Processing is intended to comprise methods of converting plant material to smaller particles either by physical manipulation of the material or by exposure to chemical or biological agents which deconstruct the plant material into smaller particles or molecules. Thus, the material can be “processed” to particles that are smaller than the starting material; however, there is no intended limit to the size of particles to be generated by “processing”. Processing further includes methods of manipulating or modifying the lignocellulosic material such that the lignocellulose fibers are separated, thus providing lignocellulosic degrading enzymes easier access to the plant fibers. It is envisioned that processing can include the liquification of the lignocellulosic plant material.

The terms “polypeptide,” “protein,” and “peptide” refer to a chain of covalently linked amino acids. Unless otherwise indicated, the term “polypeptide” encompasses both peptides and proteins. In general, the term “peptide” can refer to shorter chains of amino acids (e.g., 2-50 amino acids); however, all three terms overlap with respect to the length of the amino acid chain. Polypeptides, proteins, and peptides may comprise naturally occurring amino acids, non-naturally occurring amino acids, or a combination of both. The polypeptides, proteins, and peptides may be isolated from sources (e.g., cells or tissues) in which they naturally occur, produced recombinantly in cells in vivo or in vitro or in a test tube in vitro, or synthesized chemically. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

As used herein “nucleic acid” is a macromolecule composed of chains of monomeric nucleotides including, but not limited to deoxyribonucleic acid (DNA) and ribonucleic acid (RNA). A nucleic acid can include a gene. In particular embodiments, the nucleic acids used in the present invention are “isolated” nucleic acids.

An “isolated nucleic acid” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid molecule includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid molecule encoding at least one additional polypeptide or peptide sequence.

Accordingly, “isolated” refers to a nucleic acid molecule, nucleotide sequence, polypeptide, peptide or fragment that is altered “by the hand of man” from the natural state; i.e., that, if it occurs in nature, it has been changed or removed from its original environment, or both. For example, a naturally occurring polynucleotide or a polypeptide naturally present in a living organism in its natural state is not “isolated,” but the same polynucleotide or polypeptide separated from the coexisting materials of its natural state is “isolated”, as the term is employed herein. For example, with respect to polynucleotides, the term isolated means that it is separated from the chromosome and cell in which it naturally occurs. A polynucleotide is also isolated if separated from the chromosome and cell in which it naturally occurs in but inserted into a genetic context, chromosome or cell in which it does not naturally occur.

The term “isolated” can further refer to a nucleic acid, nucleotide sequence, polypeptide, peptide or fragment thereof that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose.

In representative embodiments of the invention an “isolated” nucleic acid, nucleotide sequence, and/or polypeptide is at least about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting plant material.

As used herein, the term “expression” (and grammatical equivalents thereof) with reference to a nucleic acid refers to transcription of the nucleic acid and optionally translation.

While a full-length enzyme can be used in the compositions; active variants and fragments thereof also can be used. As used herein, “variant” means an enzyme having a substantially similar amino acid sequence to a reference enzyme sequence. Thus, in some embodiments of the present invention, a variant means an enzyme having a substantially similar amino acid sequence to SEQ ID NO:2 and/or SEQ ID NO:4. A variant also includes a nucleic acid molecule having a substantially similar nucleotide sequence to one encoding the reference enzyme. Thus, in other embodiments of the present invention, a variant means an enzyme encoded by a nucleotide sequence substantially similar to the nucleotide sequence of SEQ ID NO:1 and/or SEQ ID NO:3.

The variant is biologically active and therefore possesses the desired activity of the reference enzyme (e.g., xylanase and cellulase activity) as described herein. The variant can result from, for example, a genetic polymorphism or human manipulation. A biologically active variant of the reference enzyme can have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the reference enzyme as determined by sequence alignment programs and parameters described elsewhere herein. An active variant can differ from the reference enzyme sequence by as few as 1-15 amino acid residues, as few as 1-10, such as 6-10, as few as 5, as few as 4, 3, 2, or even 1 amino acid residue.

Naturally occurring variants may exist within a population. Such variants can be identified by using well-known molecular biology techniques, such as the polymerase chain reaction (PCR), and hybridization as described below. Synthetically derived nucleotide sequences, for example, sequences generated by site-directed mutagenesis or PCR-mediated mutagenesis which still encode a dual activity xylanase, are also included as variants. One or more nucleotide or amino acid substitutions, additions, or deletions can be introduced into a nucleotide or amino acid sequence disclosed herein, such that the substitutions, additions, or deletions are introduced into the encoded protein. The additions (insertions) or deletions (truncations) may be made at the N-terminal or C-terminal end of the native protein, or at one or more sites in the native protein. Similarly, a substitution of one or more nucleotides or amino acids may be made at one or more sites in the native protein.

For example, conservative amino acid substitutions may be made at one or more predicted, preferably nonessential amino acid residues. A “nonessential” amino acid residue is a residue that can be altered from the wild-type sequence of a protein without altering the biological activity, whereas an “essential” amino acid is required for biological activity. A “conservative amino acid substitution” is one in which the amino acid residue is replaced with an amino acid residue with a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif, where such residues are essential for protein activity.

For example, amino acid sequence variants of the reference enzyme can be prepared by mutating the nucleotide sequence encoding the enzyme. The resulting mutants can be expressed recombinantly, and screened for those that retain biological activity by assaying for xylanase and cellulase activity using standard assay techniques. Methods for mutagenesis and nucleotide sequence alterations are known in the art. See, e.g., Kunkel (1985) Proc. Natl. Acad. Sci. USA 82:488-492; Kunkel et al. (1987) Methods in Enzymol. 154:367-382; and Techniques in Molecular Biology (Walker & Gaastra eds., MacMillan Publishing Co. 1983) and the references cited therein; as well as U.S. Pat. No. 4,873,192. Clearly, the mutations made in the DNA encoding the variant must not disrupt the reading frame and preferably will not create complimentary regions that could produce secondary mRNA structure. See, EP Patent Application Publication No. 75,444. Guidance as to appropriate amino acid substitutions that do not affect biological activity of the protein of interest may be found in the model of Dayhoff et al. (1978) Atlas of Protein Sequence and Structure (National Biomedical Research Foundation, Washington, D.C.), herein incorporated by reference.

The deletions, insertions and substitutions of the enzymes described herein are not expected to produce radical changes in the characteristics of the enzymes (e.g., the enzyme activity). However, when it is difficult to predict the exact effect of the substitution, deletion or insertion in advance of doing so, one of skill in the art will appreciate that the effect can be evaluated by routine screening assays that can screen for the particular enzyme activities of interest (e.g., xylanase and/or cellulase activity).

Assays for xylanase activity are well known in the art. See, e.g., Bailey et al. (1992) J. Biotechnol. 23:257-270; Ge et al. (2007) Anal. Biochem. 362:63-68; Gibbs et al. (1995), supra; Jeffries et al. (1998) Appl. Biochem. Biotechnol. 70-72:257-265; Kenealy & Jeffries (2003) Biotechnol. Lett. 25:1619-1623; Miller (1959) Anal. Chem. 31:426-428; Taguchi et al. (1996) Biosci. Biotechnol. Biochem. 60:983-985; Teather & Wood (1982) Appl. Environ. Microbiol. 43:777-780; and Wang & Broda (1992) Appl. Environ. Microbiol. 58:3433-3436. Likewise, kits for assaying xylanase activity are commercially available, for example, from Invitrogen (Carlsbad, Calif.) and Megazyme Int'l Ireland Ltd. (Wicklow, Ireland).

Assays for cellulase activity also are well known in the art. See, e.g., Canevascini & Gattlen (1981) Biotechnol. & Bioeng. 23:1573-1590; Chemoglazov et al. (1989) Anal. Biochem. 179:186-189; Coleman et al. (2007) Anal. Biochem. 371:146-153; Han et al. (1995) J. Biol. Chem. 270:26012-26019; Ghose (1987) Pure & Appl. Chem. 59:257-268; Miller (1959), supra; Poincelot & Day (1972) Appl. Microbiol. 23:875-879; and Sharrock (1988) J. Biochem. Biophys. Methods 17:81-105. Likewise, kits for assaying cellulase activity are commercially available, for example, from Marker Gene Technologies Inc. (Eugene. OR).

In some embodiments of the invention, variant nucleotide and amino acid sequences of the present invention also encompass sequences derived from mutagenic and recombinogenic procedures such as DNA shuffling. With such a procedure, one or more different dual activity xylanase protein coding regions can be used to create a new dual activity xylanase protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the nucleotide sequence encoding the dual activity xylanase of the invention and other known nucleotide sequences encoding dual activity xylanases to obtain a new nucleotide sequence coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

As noted above, the compositions can comprise active fragments of the dual activity enzyme. As used herein, “fragment” means a portion of the reference enzyme that retains xylanase and cellulase activity. A fragment also means a portion of a nucleic acid molecule encoding the reference enzyme. An active fragment of the enzyme can be prepared, for example, by isolating a portion of an enzyme-encoding nucleic acid molecule that expresses the encoded fragment of the enzyme (e.g., by recombinant expression in vitro), and assessing the activity of the fragment. Nucleic acid molecules encoding such fragments can be at least about 150, 200, 250, 300, 350, 400, 450, 500, 550, 600, 650, 700, 800, 900, 1,000, 1,100, 1,200, 1,300 or 1,400 contiguous nucleotides, or up to the number of nucleotides present in a full-length enzyme-encoding nucleic acid molecule. As such, polypeptide fragments can be at least about 50, 60, 70, 80, 90, 100, 125, 150, 175, 200, 225 or 250 contiguous amino acid residues, or up to the total number of amino acid residues present in the full-length enzyme.

As used herein, “sequence identity” or “identity” in the context of two nucleotide or amino acid sequences means that the residues in the two sequences that are the same when aligned for maximum correspondence over a specified region. When percentage of sequence identity is used in reference to proteins such as enzymes it is recognized that residue positions that are not identical often differ by conservative amino acid substitutions, where amino acid residues are substituted for other amino acid residues with similar chemical properties (e.g., charge or hydrophobicity) and therefore do not change the functional properties of the enzyme. When sequences differ in conservative substitutions, the percent sequence identity can be adjusted upwards to correct for the conservative nature of the substitution. Sequences that differ by such conservative substitutions are said to have “sequence similarity” or “similarity.” Means for making this adjustment are well known to one of skill in the art. The scoring of conservative substitutions is calculated, for example, as implemented in the PC/GENE Program.

As used herein, “percentage of sequence identity” means a value determined by comparing two optimally aligned sequences over a defined region, wherein the portion of the nucleotide or amino acid sequence in the defined region may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the window of comparison, and multiplying the result by 100 to yield the percentage of sequence identity.

Determining percent sequence identity between any two sequences can be accomplished using a mathematical algorithm. Non-limiting examples of such mathematical algorithms are the algorithm of Myers & Miller (1988) CABIOS 4:11-17; the local alignment algorithm of Smith et al. (1981) Adv. Appl. Math. 2:482-489; the global alignment algorithm of Needleman & Wunsch (1970) J. Mol. Biol. 48:443-453; the search-for-local alignment method of Pearson & Lipman (1988) Proc. Natl. Acad. Sci. USA 85:2444-2448; the algorithm of Karlin & Altschul (1990) Proc. Natl. Acad. Sci. USA 87:2264-2268, modified as in Karlin & Altschul (1993) Proc. Natl. Acad. Sci. USA 90:5873-5877.

Computer implementations of these mathematical algorithms can be used to compare sequences to determine sequence identity. Such implementations include, but are not limited to, CLUSTAL in the PC/Gene Program (Intelligenetics; Mountain View, Calif.); the ALIGN Program (Version 2.0; Corpet et al. (1988) Nucleic Acids Res. 16:10881-10890; Higgins et al. (1988) Gene 73:237-244; Higgins et al. (1989) CABIOS 5:151-153; Huang et al. (1992) CABIOS 8:155-165; and Pearson et al. (1994) Meth. Mol. Biol. 24:307-331) and GAP, BESTFIT, BLAST, FASTA and TFASTA in the GCG Wisconsin Genetics Software Package, Version 10 (Accelrys Inc.; San Diego, Calif.). Alignments using these programs can be performed using the default parameters.

Accordingly, the dual activity enzymes can be provided in the composition as a partially or fully purified full-length enzyme, or as an active variant or fragment thereof, or even can be provided directly as an enzyme-producing microorganism, plant, plant part or plant cell (e.g., an organism transformed with a nucleotide encoding a dual activity enzyme of the invention). Complete purification is not required in any case. The enzyme, variant or fragment therefore can be at least about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% pure (w/w), or more.

Methods of purifying polypeptides and proteins such as enzymes are well known in the art. See, e.g., Cesar & Mr\check{s}a (1996) Enzyme Microb. Tech. 19:289-296; Chivero et al. (2001) Food Chem. 72:179-185; de Albuquerque Lucena-Neto et al. (2004) Br. J. Microb. 35:86-90; Ehle & Horn (1990) Bioseparation 1:97-110; Gupta et al. (2002) Biotechnol. Lett. 24:2005-2009; Hengen (1995) Trends Biochem Sci. 20:285-286; Kanda et al. (1985) J. Biochem. 98:1545-1554; Khasin et al. (1993) Appl. Environ. Microb. 59:1725-1730; Kudo et al. (1985) J. Gen. Microbiol. 131:2825-2830; Regnier (1983) Science 222:245-252; Roy & Uddin (2004) Pak. Biol. Sci. 7:372-379; Royer & Nakas (1991) Eu. J. Biochem. 202:521-529; Sá-Pereira et al. (2003) Mol. Biotechnol. 24:257-281; Shaw, “Peptide purification by reverse-phase HPLC” 257-287 In: Methods in Molecular Biology, Vol. 32 (Walker ed., Humana Press 1994); Simpson et al. (1991) Biochem. J. 277:419-417; Encyclopedia of Chemical Technology, 52^{nd} ed. (Kirk-Othmer ed., Wiley-Interscience 2007); Basic Methods in Protein Purification and Analysis: A Laboratory Manual (Simpson et al. eds., Cold Spring Harbor Laboratory Press 2008); Enzyme Purification and Related Techniques. Methods in Enzymology, Vol. 22 (Jakoby ed., Academic Press Inc. 1971) Methods in Enzymology: Affinity Techniques—Enzyme Purification: Part B: Vol. 34: Affinity Techniques Part B (Kaplan et al. eds., Elsevier 1974); as well as US Patent Application Publication Nos. 2009/0137022 and 2009/0239262; and U.S. Pat. Nos. 4,347,322; 4,634,673; 4,725,544 and 5,437,992.

Examples of purification techniques suitable for enzymes include, but are not limited to, precipitation such as ammonium sulfate precipitation, separation based on molecular size such as gel filtration, separation based on charge such as ion-exchange chromatography, separation based on specific interaction with other biomolecules such as bio-affinity chromatography or antibody recognition of amino acid sequence, separation based on other principles such as hydrophobic interaction chromatography or hydroxyapatite chromatography and separation based on electrophoretic principles (e.g., acrylamide, starch electrophoresis).

As described below, in some embodiments, nucleotide sequences encoding the enzymes of the present invention (e.g., SEQ ID NO:1 and/or SEQ ID NO:3), or biologically active fragments or variants thereof, are transformed into a plant, plant part or plant cell and the transformed plant, plant part or plant cell comprising said enzymes is used as the source of the lignocellulosic material, thereby providing the dual activity enzyme in the lignocellulosic material itself.

Full length dual activity enzyme of the present invention, or an active variant or fragment thereof, can be used in the methods and compositions of the present invention and can be provided in an amount from about 0.01 units to about 1000 units, about 0.1 units to about 500 units or about 1 unit to about 50 units of enzyme activity per gram dry weight of lignocellulosic material to be treated. Thus, in some embodiments, a suitable enzyme dosing can be about 0.01 units to 1000 units per gram of dry lignocellulosic material. In other embodiments, a suitable enzyme dose is 0.1 units to 500 units per gram of dry lignocellulosic material. Alternatively, in further embodiments of the invention, the compositions can include about 0.01 unit to about 1 unit, about 1 unit to about 10 units, about 10 units to about 100 units, about 100 units to about 200 units, about 200 units to about 300 units, about 300 units to about 400 units, about 400 units to about 500 units, about 500 units to about 600 units, about 600 units to about 700 units, about 700 units to about 800 units, about 800 units to about 900 units, about 900 units to about 1000 units, or more of enzyme activity per gram dry weight of lignocellulosic material to be treated. One unit can be defined as an amount of enzyme required to liberate 1 mmol of xylose per minute at an assay temperature. Alternatively, one unit can be defined as an amount of enzyme required to liberate 1 μmol of cellulose per minute at an assay temperature.

The activity of the enzyme composition can be determined as follows: to 0.5 ml of xylan or cellulose solution (1%; Sigma, St. Louis, Mo.; prepared in a 50 mM phosphate buffer, pH 7) add 0.5 ml of suitably diluted enzyme in the same buffer. The solution can be incubated at 70° C. for about 10 minutes. The reaction then can be stopped by adding 1 ml DNS reagent (3,5-dinitrosalicylate 10 g/L; Na/K tartrate 300 g/L; NaOH 16 g/L), and the color can be developed by boiling the sample for 5 minutes (DNS assay). See, e.g., Ghose (1987), supra; and Miller (1959), supra. The absorbency then can be measured at a wavelength of 540 nm. One enzyme unit liberates one μmol of reducing sugars calculated as xylose per minute or cellulose per minute under assay conditions. The activity can be calculated from an enzyme dilution liberating four μmol of reducing sugar under assay conditions.

If the activity of the enzyme composition is insufficient, it can be concentrated prior to use. Methods of concentrating polypeptides and proteins such as enzymes are well known in the art. See, e.g., Ahmed, Principles and Reactions of Protein Extraction, Purification, and Characterization (CRC Press 2004); Dennison, A Guide to Protein Isolation, 2^{nd} ed. (Kluwer Academic Publishers 2003); Degerli & Akpinar (2001) Anal. Biochem. 297:192-194; and Protein Methods, 2^{nd} ed. (Bollag et al. eds., Wiley Publishers 1996). For example, ultrafiltration can be used to concentrate a supernatant of the enzyme 5-25 fold.

The compositions may comprise the dual activity protein or enzyme, variant or fragment thereof only, or can be a mixture (i.e., “cocktail”) having the dual activity enzyme (e.g., SEQ ID NO:2 and/or 4), variant or fragment thereof and at least one or more other enzyme(s) including, but not limited to, other lignocellulolytic enzymes useful in processing lignocellulosic materials such as other cellulases, hemicellulases, ligninases, pectinases and proteases. As used herein, “lignocellulolytic enzyme” means an enzyme that processes lignocellulosic material such as cellulose, hemicellulose and lignin, as well as other polysaccharide and protein components that may be a part of or associated with lignocellulose.

The at least one or more other enzyme(s) can be provided to the lignocellulosic material in the form of a plant, plant part, or plant cell, transformed with one or more nucleotide sequences encoding the one or more other enzymes. The transformed plant, plant part, or plant cell can be the same or different from the plant, plant part or plant cell transformed with a nucleotide sequence encoding the dual activity enzyme of the present invention. Thus, in some embodiments, a plant, plant part or plant cell can be transformed with more than one nucleotide sequence encoding various enzymes useful in the processing of lignocellulolytic material.

Non-limiting examples of cellulases include mannan endo-1,4-β-mannosidase, 1,3-β-D-glucan glucanohydrolase, 1,3-β-glucan glucohydrolase, 1,3-1,4-β-D-glucan glucanohydrolase and 1,6-β-D-glucan glucanohydrolase.

Examples of hemicellulases include, but are not limited to, α-L-arabinofuranosidase, α-glucuronidase, acetyl mannan esterase, acetyl xylan esterase, α-galactosidase, β-glucosidase, β-1,4-xylosidase, endo-galactanase, endo-β-1,4-mannanase, endo-α-1,5-arabinanase, exo-β-1,4-mannosidase, exo-β-1,4-xylosidase, feruloyl esterase, ferulic acid esterase, p-cumaric acid esterase, glucuronoxylan xylanohydrolase and xyloglucan endotransglycosylase.

Examples of ligninases include, but are not limited to, diarylpropane peroxidase, glucose oxidase, glyoxal oxidase, lignin peroxidase (LiP), manganese peroxidase, methanol oxidase, methanol oxidoreductase, phenol oxidase (laccase), phenol peroxidase and veratryl alcohol oxidase.

Examples of pectinases include, but are not limited to, pectolyase, pectozyme and polygalacturonase.

Examples of proteases include, but are not limited to, asclepain, bromelain, caricain, chymopapain, collagenase, glycyl endopeptidase, pepsin, pronase, subtilisin and thermolysin.

The compositions also can include other enzymes such as amylase, catalase, cutinase, glucanase, glucoamylase, glucose isomerase, lipase, phytase, pullulanase and xylose isomerase.

As above, any of these enzymes can be provided as partially or fully purified full-length enzymes, or active variants or fragments thereof, or can be provided as an enzyme-producing microorganism. Moreover, any of these enzymes can be provided in an amount effective to hydrolyze their substrate, such as in amounts from about 0.001 wt. % to about 50 wt. %, from about 0.01 wt. % to about 50 wt. %, from about 0.1 wt. % to about 50 wt. %, from about 1 wt. % to about 50 wt. %, from about 10 wt. % to about 50 wt. %, from about 20 wt. % to about 50 wt. %, from about 30 wt. % to about 50 wt. %, from about 40 wt. % to about 50 wt. %, or more.

The compositions also can include agents typically used in processing lignocellulosic materials such as a chlorine, detergent, hypochlorite, hydrogen peroxide, oxalic acid, peracid, pH-regulating agent, trisodium phosphate, sodium chlorite, sodium nitrate, surfactant, urea and water.

Examples of detergents include, but are not limited to, anionic, cationic or neutral detergents such as Nonidet (N)P-40, sodium dodecyl sulfate (SDS), sodium lauryl sulfate (SLS), sulfobetaine, n-octylglucoside, deoxycholate, Triton® X-100 (Dow Chemical Co.; Midland, Mich.) and Tween® 20 (ICI Americas, Inc.; Bridgewater, N.J.).

Non-limiting examples of peracids include, but are not limited to, meta-chloroperoxybenzoic acid, perbenzoic acid, perchloric acid, perphthalic acid, permaleic acid, peracetic acid, performic acid, perproprionic acid and p-nitro perbenzoic acid.

Non-limiting examples of surfactants include a secondary alcohol ethoxylate, fatty alcohol ethoxylate, nonylphenol ethoxylate, phosphate ester of fatty alcohols, polyoxyethylene ether, polyethylene glycol, polyoxyethylenated alkyl phenol and stearic acid and tridecyl ethoxylate.

Any of these agents can be provided as partially or fully purified. Moreover, any of these agents can be provided in an amount from about 0.001 wt. % to about 50 wt. %, from about 0.01 wt. % to about 50 wt. %, from about 0.1 wt. % to about 50 wt. %, from about 1 wt. % to about 50 wt. %, from about 10 wt. % to about 50 wt. %, from about 20 wt. % to about 50 wt. %, from about 30 wt. % to about 50 wt. %, from about 40 wt. % to about 50 wt. %, or more.

As described above, the compositions of the present invention also can include enzyme producing microorganisms, especially ethanologenic and/or lignin-solubilizing microorganisms. Non-limiting examples of ethanologenic and or lignin-solubilizing microorganisms include bacteria and yeast. See generally, Burchhardt & Ingram (1992) Appl. Environ. Microbiol. 58:1128-1133; Dien et al. (1998) Enzyme Microb. Tech. 23:366-371; Keating et al. (2004) Enzyme Microb. Tech. 35:242-253; Lawford & Rousseau (1997) Appl. Biochem. Biotechnol. 63-65:221-241; Handbook on Bioethanol: Production and Utilization (Wyman ed., CRC Press 1996); as well as US Patent Application Publication Nos. 2009/0246841 and 2009/0286293; and U.S. Pat. No. 6,333,181. Such microorganisms can produce enzymes that assist in processing lignocellulosic material such as alcohol dehydrogenase, pyruvate decarboxylase, transaldolase, transketolasepyruvate decarboxylase, xylose reductase, xylitol dehydrogenase or xylose isomerase xylulokinase. In some embodiments of the invention, the ethanologenic and or lignin-solubilizing microorganisms include, but are not limited to, members in genera such as Candida, Erwinia, Escherichia, Klebsiella, Pichia, Saccharomyces, Streptomyces and Zymomonas. See, e.g., Dien (1998), supra; Ingram & Conway (1988) Appl. Environ. Microbiol. 54:397-404; Jarboe et al. (2007) Adv. Biochem. Engin./Biotechnol. 108:237-261; Keating et al. (2004) J. Indust. Microbiol. Biotech. 31:235-244; Keating et al. (2006) Biotechnol. Bioeng. 93:1196-1206; Pasti et al. (1990) Appl. Environ. Microbiol. 56:2213-2218; and Zhang et al. (1995) Science 267:240-243.

The compositions comprising the dual activity enzymes of the present invention, active variant or fragment thereof, as well as any of the other enzymes and/or agents described above, can be prepared as a liquid, slurry, solid or gel.

Transgenic Organisms

In some embodiments, the present invention provides compositions comprising plants, plant parts, and plant cells transformed with a nucleotide sequence encoding at least one dual activity enzyme, or an active variant or fragment thereof having xylanase and cellulase activity (e.g., an enzyme having the amino acid sequence of SEQ ID NO:2 and/or SEQ ID NO:4, or an active fragment or active variant of SEQ ID NO:2 and/or SEQ ID NO:4). As used herein, “plant” means any plant including woody and non-woody plants. In some embodiments, the plant is a seed plant (i.e., an angiosperm and/or gymnosperm). As used herein, the term “plant part” includes but is not limited to embryos, pollen, ovules, seeds, leaves, flowers, branches, fruit, kernels, ears, cobs, husks, stalks, roots, root tips, anthers, plant cells, plant protoplasts, plant tissues, plant cell tissue cultures, plant calli, plant clumps, and plant cells that are intact in plants or parts of plants, and the like. Further, as used herein, “plant cell” refers to a structural and physiological unit of the plant, which comprises a cell wall and also may refer to a protoplast. A plant cell of the present invention can be in the form of an isolated single cell or can be a cultured cell or can be a part of a higher-organized unit such as, for example, a plant tissue or a plant organ.

In particular embodiments of the present invention, the transformed plants are used in the processing of lignocellulosic plant material (e.g., the production of cellulosic ethanol; production of biofuels; production of fermentable sugars from lignocellulosic biomass; processing of fruit and/or vegetable material; processing or treating pulp; and the like). In some embodiments, the transgenic plants of the invention can serve as the source of xylanase and cellulase enzyme activities in the lignocellulose conversion process (i.e., the processing of the lignocellulosic material). The transformed plant, plant part or plant cell can be used directly or the enzymes can be isolated from the transformed plant, plant part or plant cell.

In further embodiments, the transgenic plants of the invention can be used as the sole source of lignocellulosic material or they can be used in combination with other sources of lignocellulosic material, both transformed and non-transformed sources. Thus, in some embodiments, the transgenic plants, plant parts, or plant cells of the present invention are combined with lignocellulosic materials derived from non-transgenic plants, plant parts and/or plant tissues. In other embodiments, the transgenic plants of the invention can be used in combination with other transgenic plants expressing other enzymes useful in the processing of lignocellulosic material, for example AMY797E, a transgenic plant expressing a thermostable α-amylase. In some embodiments, the transgenic plants, plant parts and/or plant cells of the present invention can be transformed with nucleic acids encoding additional enzymes useful in the processing of lignocellulosic material (in addition to the dual activity enzymes of the present invention, e.g., in addition to an enzyme having the amino acid sequence of SEQ ID NO:2 and/or SEQ ID NO:4), which transgenic plants, plant parts and/or plant tissues can then be used as a source of multiple enzymes useful in the processing of the lignocellulosic material.

Plants of the present invention can include any plant. Non-limiting examples of plants of the present invention include barley, beans in general, Brassica spp., clover, cocoa, coffee, cotton, flax, maize, millet, peanut, rape/canola, rice, rye, safflower, sorghum, soybean, sugarcane, sugar beet, sunflower, sweet potato, tea and wheat; vegetables such as broccoli, brussel sprouts, cabbage, carrot, cassaya, cauliflower, cucurbits, lentils, lettuce, pea, peppers, potato, radish and tomato; grasses such as alfafa, bermudagrass, elephantgrass, rhodesgrass, tall fescue grass, tall wheat grass, Miscanthus spp. and switchgrass; tree fruits such as apples, apricots, avocado, banana, citrus, coconuts, pears, peaches, pineapple and walnuts; and flowers such as carnations, orchids, roses, and any combination thereof.

In some embodiments, a signal sequence or targeting sequence can be used to target the enzyme to organelles such as the vacuole, the endoplasmic reticulum, the chloroplast, and the like. In this manner, a signal or targeting sequence is operably linked to the dual activity enzyme. Therefore, a nucleic acid construct or expression cassette for expression of the enzyme in a plant will include a signal sequence operably linked to the coding sequence for the dual activity enzyme. Such targeting sequences will target the dual activity enzyme to a vacuole, the endoplasmic reticulum, the chloroplast, and the like.

Examples of such sequences include, but are not limited to, the transit peptide for the acyl carrier protein, the small subunit of RUBISCO, plant EPSP synthase, and the like. See, e.g. Archer et al. (1990) J. Bioenerg. Biomemb. 22:789-810); Clark et al. (1989) J. Biol. Chem. 264:17544-17550; Daniell (1999) Nat. Biotech. 17:855-856; de Castro Silva Filho et al. (1996) Plant Mol. Biol. 30:769-780; Della-Cioppa et al. (1987) Plant Physiol. 84:965-968; Lamppa et al. (1988) J. Biol. Chem. 263:14996-14999; Lawrence et al. (1997) J. Biol. Chem. 272:20357-20363; Romer et al. (1993) Biochem. Biophys. Res. Commun. 196:1414-1421; Schmidt et al. (1993) J. Biol. Chem. 268:27447-27457; Schnell et al. (1991) J. Biol. Chem. 266:3335-3342); Shah et al. (1986) Science 233:478-481; Von Heijne et al. (1991) Plant Mol. Biol. Rep. 9:104-126; and Zhao et al. (1995) J. Biol. Chem. 270:6081-6087; as well as U.S. Pat. No. 6,338,168.

Endoplasmic reticulum signal peptides include those described in Raikhel & Chrispeels “Protein sorting and vesicle traffic” In: Biochemistry and Molecular Biology of Plants (Buchanan et al. eds., American Society of Plant Physiologists 2000). See also, Denecke et al. (1992) EMBO J. 11:2345-2355; Denecke et al. (1993) J. Exp. Bot. 44:213-221; Gomord et al. (1996) Plant Physiol. Biochem. 34:165-181; Lehmann et al. (2001) Plant Physiol. 127:436-449; Munro & Pelham (1986) Cell 46:291-300; Munro & Pelham (1987) Cell 48:899-907; Vitale et al. (1993) J. Exp. Bot. 44:1417-1444; and Wandelt et al. (1992) Plant J. 2:181-192.

In some instances, it may be desirable to localize the dual activity enzyme within a vacuole. For vacuole-targeted expression of dual activity enzymes, plants are transformed with vectors that include a vacuolar targeting sequence such as that from a tobacco chitinase gene. Vacuole signal sequences (i.e., vacuolar sorting sequences) include, but are not limited to, the barley polyamino oxidase 2 (BPAO2) signal sequence (Cervelli et al. (2004) The Plant Journal 40:410-418). For other vacuolar sorting signals, see U.S. application Ser. No. 12/359,421 and Raikhel & Chrispeels (2000), supra.

Plastid-targeting sequences are well known in the art and can be used in the practice of the invention. See, e.g., Clark et al. (1989) J. Biol. Chem. 264:17544-17550; Della-Cioppa et al. (1987), supra; Romer et al. (1993) Biochem. Biophys. Res. Commun. 196:1414-1421; Shah et al. (1986) Science 233:478-481; and Von Heijne et al. (1991) Plant Mol. Biol. Rep. 9:104-126. Additional chloroplast-targeting sequences include the chloroplast small subunit of ribulose-1,5-bisphosphate carboxylase (Rubisco) (de Castro Silva Filho et al. (1996) Plant Mol. Biol. 30:769-780; and Schnell et al. (1991) J. Biol. Chem. 266:3335-3342); 5-(enolpyruvyl)shikimate-3-phosphate synthase (EPSPS) (Archer et al. (1990), supra); tryptophan synthase (Zhao et al. (1995), supra); plastocyanin (Lawrence et al. (1997), supra); chorismate synthase (Schmidt et al. (1993), supra); and the light harvesting chlorophyll a/b binding protein (LHBP) (Lamppa et al. (1988), supra). One of skill in the art also can envision generating transgenic plants in which the chloroplasts have been transformed to overexpress the dual activity enzyme. See, e.g., Daniell (1999) Nat. Biotech. 17:855-856; as well as U.S. Pat. No. 6,338,168.

Methods for transforming chloroplasts are well known in the art. See, e.g., Svab et al. (1990) Proc. Natl. Acad. Sci. USA 87:8526-8530; Svab & Maliga (1993) Proc. Natl. Acad. Sci. USA 90:913-917; Svab & Maliga (1993) EMBO J. 12:601-606. Likewise, nucleotide sequences to be targeted to the chloroplast can be optimized for expression in the chloroplast to account for differences in codon usage between the plant nucleus and this organelle. In this manner, the polynucleotide of interest may be synthesized using chloroplast-preferred codons. See, e.g., U.S. Pat. No. 5,380,831.

For localization of the dual activity enzyme within the apoplast, an apoplast-targeting sequence such as a maize γ-zein N-terminal signal sequence (U.S. Pat. No. 7,102,057) can be used.

Additional guidance on targeting polypeptides can be found, for example, in Bruce (2001) Biochim Biophys Acta 1541:2-21; Emanuelsson et al. (2000) J. Mol. Biol. 300:1005-1016; Emanuelsson & von Heijne (2001) Biochim Biophys Acta 1541:114-119; Hadlington & Denecke (2000) Curr. Opin. Plant Biol. 3:461-468; Nicchitta (2002) Curr. Opin. Cell Biol. 14:412-416; and Silva-Filho (2003) Curr. Opin. Plant Biol. 6:589-595.

Briefly, methods of expressing a nucleotide sequence in a plant, plant part or plant cell include transforming a plant cell with, for example, a nucleic acid construct such as an expression cassette having a coding sequence for a dual activity enzyme, variant or fragment thereof, operably linked to one or more regulatory sequences. Coding sequence for some embodiments of the dual activity enzyme include the nucleotide sequence of SEQ ID NO:1 and/or SEQ ID NO:3, or a fragment or variant of SEQ ID NO:1 and/or SEQ ID NO:3, or the amino acid sequence of SEQ ID NO:2 and/or SEQ ID NO:4, or a fragment or variant of SEQ ID NO:2 and/or SEQ ID NO:4. The transformed plant, plant part or plant cell is grown under conditions where the nucleotide sequence is expressed in the plant cell. In some embodiments, the methods include an additional step of regenerating the transformed plant cell into a morphologically normal, fertile plant.

A plant, plant part or plant cell expressing the dual activity enzyme therefore can be obtained by introducing into the plant, plant part or plant cell a coding sequence for a dual activity enzyme. In some embodiments, the coding sequence can be present in an expression cassette. In other embodiments, the coding sequence can be present in a nucleic acid construct such as a vector.

As used herein, “expression cassette” means a nucleic acid molecule having at least a control sequence operably linked to a coding sequence of interest (e.g., the dual activity enzyme). In this manner, plant promoters in operable interaction with the nucleotide sequences for the dual activity enzyme are provided in expression cassettes for expression in a plant, plant part or plant cell.

Expression of the coding sequence of interest in an expression cassette can be under the control of a constitutive promoter or an inducible promoter that initiates transcription only when the plant, plant part or plant cell is exposed to some particular external stimulus. Alternatively, the expression cassette can be under the control of a tissue-specific promoter that functions in a particular tissue, organ or even at a particular stage of development.

For purposes of the invention, the regulatory regions (i.e., promoters, transcriptional regulatory regions, and translational termination regions) may be native/analogous to the plant, plant part or plant cell and/or the regulatory regions may be native/analogous to each other (i.e., other regulatory regions). Alternatively, the regulatory regions may be heterologous to the plant (or plant part or plant cell) and/or to each other.

As used herein, “heterologous” in reference to a sequence means a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The choice of promoters to be used depends upon several factors, including, but not limited to, cell- or tissue-specific expression, desired expression level, efficiency, inducibility and selectability. For example, where expression in a specific tissue or organ is desired, a tissue-specific promoter can be used. In contrast, where expression in response to a stimulus is desired, an inducible promoter can be used. Where continuous expression is desired throughout the cells of a plant, a constitutive promoter can be used. It is a routine matter for one of skill in the art to modulate the expression of a nucleotide sequence by appropriately selecting and positioning promoters and other regulatory regions relative to that sequence.

Therefore, in some instances, constitutive promoters can be used. Examples of constitutive promoters include, but are not limited to, the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci. USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter.

Moreover, tissue-specific regulated nucleic acids and/or promoters have been reported in plants. Thus, in some embodiments, tissue specific promoters can be used. Some reported tissue-specific nucleic acids include those encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Thus, the promoters associated with these tissue-specific nucleic acids can be used in the present invention. Additional examples of tissue-specific promoters include, but are not limited to, the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” 29-39 In: Genetic Engineering of Plants (Hollaender ed., Plenum Press 1983); and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). Particularly useful for seed-specific expression is the pea vicilin promoter (Czako et al. (1992) Mol. Gen. Genet. 235:33-40; as well as U.S. Pat. No. 5,625,136). Other useful promoters for expression in mature leaves are those that are switched on at the onset of senescence, such as the SAG promoter from Arabidopsis (Gan et al. (1995) Science 270:1986-1988).

In some instances, inducible promoters can be used. Examples of inducible promoters include, but are not limited to, tetracycline repressor system promoters, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters. Other inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108.

In addition to the promoters described above, the expression cassette also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences and polyadenylation signal sequences.

The expression cassette also can optionally include a transcriptional and/or translational termination region (i.e., termination region) that is functional in plants. A variety of transcriptional terminators are available for use in expression cassettes and are responsible for the termination of transcription beyond the transgene and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and the pea rbcs E9 terminator. These can be used in both monocotyledons and dicotyledons. In addition, a coding sequence's native transcription terminator can be used.

As discussed above, a signal sequence may be operably linked to the dual activity enzyme of the present invention to direct the enzyme into a cellular compartment. In this manner, the expression cassette will comprise a coding sequence for the dual activity enzyme operably linked to a nucleic acid sequence for the signal sequence. The signal sequence may be operably linked at the N- or C-terminus of the enzyme.

Regardless of the type of regulatory sequence(s) used, they can be operably linked to the coding sequence of the dual activity enzyme. As used herein, “operably linked” means that elements of a nucleic acid construct such as an expression cassette are configured so as to perform their usual function. Thus, control sequences (i.e., promoters) operably linked to a coding sequence are capable of effecting expression of the coding sequence. The control sequences need not be contiguous with the coding sequence, so long as they function to direct the expression thereof. Thus, for example, intervening untranslated, yet transcribed, sequences can be present between a promoter and a coding sequence, and the promoter sequence can still be considered “operably linked” to the coding sequence.

The expression cassette also can include a nucleotide sequence for a selectable marker, which can be used to select a transformed plant, plant part or plant cell. As used herein, “selectable marker” means a nucleic acid that when expressed imparts a distinct phenotype to the plant, plant part or plant cell expressing the marker and thus allows such transformed plants, plant parts or plant cells to be distinguished from those that do not have the marker. Such a nucleic acid may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleic acid encoding neo or nptII, which confers resistance to kanamycin, G418, and the like (Potrykus et al. (1985) Mol. Gen. Genet. 199:183-188); a nucleic acid encoding bar, which confers resistance to phosphinothricin; a nucleic acid encoding an altered 5-enolpyruvylshikimate-3-phosphate (EPSP) synthase, which confers resistance to glyphosate (Hinchee et al. (1988) Biotech. 6:915-922); a nucleic acid encoding a nitrilase such as bxn from Klebsiella ozaenae that confers resistance to bromoxynil (Stalker et al. (1988) Science 242:419-423); a nucleic acid encoding an altered acetolactate synthase (ALS) that confers resistance to imidazolinone, sulfonylurea or other ALS-inhibiting chemicals (EP Patent Application No. 154204); a nucleic acid encoding a methotrexate-resistant dihydrofolate reductase (DHFR) (Thillet et al. (1988) J. Biol. Chem.; 263:12500-12508); a nucleic acid encoding a dalapon dehalogenase that confers resistance to dalapon; a nucleic acid encoding a mannose-6-phosphate isomerase (also referred to as phosphomannose isomerase (PMI)) that confers an ability to metabolize mannose (U.S. Pat. Nos. 5,767,378 and 5,994,629); a nucleic acid encoding an altered anthranilate synthase that confers resistance to 5-methyl tryptophan; and/or a nucleic acid encoding hph that confers resistance to hygromycin. One of skill in the art is capable of choosing a suitable selectable marker for use in an expression cassette.

Additional selectable markers include, but are not limited to, a nucleic acid encoding β-glucuronidase or uidA (GUS) that encodes an enzyme for which various chromogenic substrates are known; an R-locus nucleic acid that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleic acid encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleic acid encoding xylE that encodes a catechol dioxygenase (Zukowsky et al. (1983) Proc. Natl. Acad. Sci. USA 80:1101-1105); a nucleic acid encoding tyrosinase, an enzyme capable of oxidizing tyrosine to DOPA and dopaquinone, which in turn condenses to form melanin (Katz et al. (1983) J. Gen. Microbiol. 129:2703-2714); a nucleic acid encoding β-galactosidase, an enzyme for which there are chromogenic substrates; a nucleic acid encoding luciferase (lux) that allows for bioluminescence detection (Ow et al. (1986) Science 234:856-859); a nucleic acid encoding aequorin which may be employed in calcium-sensitive bioluminescence detection (Prasher et al. (1985) Biochem. Biophys. Res. Comm. 126:1259-1268); or a nucleic acid encoding green fluorescent protein (Niedz et al. (1995) Plant Cell Reports 14:403-406).

An expression cassette of the present invention also can include coding sequences for other lignocellulolytic enzymes. Such sequences can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, the nucleotide sequences of interest can be combined at any time and in any order. For example, a transgenic plant comprising one or more desired traits can be used as the target to introduce further traits by subsequent transformation. The nucleotide sequences can be introduced simultaneously in a co-transformation protocol with coding sequences for the dual activity enzyme provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

The expression cassette also can include a coding sequence for one or more polypeptides for agronomic traits that primarily are of benefit to a seed company, grower or grain processor, for example, bacterial pathogen resistance, fungal resistance, herbicide resistance, insect resistance, nematode resistance and virus resistance. See, e.g., U.S. Pat. Nos. 5,304,730; 5,495,071; 5,569,823; 6,329,504 and 6,337,431. The trait also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various traits of interest, as well as methods for introducing these traits into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Application Publication No. 2001/0016956. See also, on the World Wide Web at lifesci.sussex.ac.uk/home/Neil_Crickmore/Bt/.

Numerous nucleotide sequences are known to enhance expression from within a transcriptional unit, and these sequences can be used in conjunction with the coding sequence for the dual activity enzyme to increase its expression in transgenic plants. For example, introns of the maize Adhl gene and Intron 1 have been shown to enhance gene expression. See, e.g., Callis et al. (1987) Genes Develop. 1:1183-1200.

Likewise, a number of non-translated leader sequences derived from viruses also are known to enhance gene expression. Specifically, leader sequences from Tobacco Mosaic Virus (TMV, the “omega-sequence”), Maize Chlorotic Mottle Virus (MCMV) and Alfalfa Mosaic Virus (AMV) have been shown to be effective in enhancing expression (Gallie et al. (1987) Nucleic Acids Res. 15:8693-8711; and Skuzeski et al. (1990) Plant Mol. Biol. 15:65-79). Other leader sequences known in the art include, but are not limited to, picornavirus leaders such as an Encephalomyocarditis (EMCV) 5′ noncoding region leader (Elroy-Stein et al. (1989) Proc. Natl. Acad. Sci. USA 86:6126-6130); potyvirus leaders such as a Tobacco Etch Virus (TEV) leader (Allison et al. (1986) Virology 154:9-20); Maize Dwarf Mosaic Virus (MDMV) leader (Allison et al. (1986), supra); human immunoglobulin heavy-chain binding protein (BiP) leader (Macejak & Samow (1991) Nature 353:90-94); untranslated leader from the coat protein mRNA of AMV (AMV RNA 4; Jobling & Gehrke (1987) Nature 325:622-625); tobacco mosaic TMV leader (Gallie et al. (1989) Molecular Biology of RNA 237-256); and MCMV leader (Lommel et al. (1991) Virology 81:382-385). See also, Della-Cioppa et al. (1987) Plant Physiol. 84:965-968.

Once the coding sequence for the dual activity enzyme is cloned into the expression cassette, it can be transformed into, for example, a plant, plant part or plant cell.

As used herein, “transformation” means introducing a nucleic acid molecule or fragment into a plant, plant part or plant cell. Cells containing the transformed nucleic acid molecule are referred to as “transgenic” cells, and organisms comprising transgenic cells are referred to as “transgenic organisms.”

“Introducing” in the context of a nucleotide sequence of interest means presenting to the plant the nucleotide sequence in such a manner that the nucleotide sequence gains access to the interior of a cell of a plant or plant part. Where more than one nucleotide sequence is to be introduced, these nucleotide sequences can be assembled as part of a single polynucleotide or nucleic acid construct, or as separate polynucleotide or nucleic acid constructs, and can be located on the same or different transformation vectors. Further, these polynucleotides can be introduced into plant cells in a single transformation event, in separate transformation events, or, e.g., as part of a breeding protocol.

As used herein, “transient transformation” or “transiently transformed” in the context of a nucleic acid molecule means that the nucleic acid molecule is introduced into the cell and does not integrate into the genome of the cell.

By “stably introducing” or “stably introduced” in the context of a nucleic acid molecule introduced into a cell is intended that the introduced nucleic acid molecule is stably incorporated into the genome of the cell, and thus the cell is stably transformed with the nucleic acid molecule.

“Stable transformation” or “stably transformed” as used herein means that a nucleic acid molecule that is introduced into a cell and integrates into the genome of the cell. As such, the integrated nucleic acid molecule is capable of being inherited by the progeny thereof, more particularly, by the progeny of multiple successive generations. The genome as used herein also includes the plastid genome, and therefore includes integration of the nucleic acid molecule into, for example, the chloroplast genome. Stable transformation as used herein can also refer to a transgene that is maintained extrachromosomally, for example, as a minichromosome.”

Thus, in some embodiments of the present invention, a plant, plant part or plant cell is transiently transformed. In other embodiments of the present invention, a plant, plant part or plant cell is stably transformed. Thus, in further embodiments, wherein a plant part or plant cell is stably transformed, a stably transformed plant can be regenerated from said stably transformed plant part or plant cell.

Methods for introducing nucleotide sequences into plants are known in the art including, but not limited to, transient transformation methods, stable transformation methods, and virus-mediated methods. Some non-limiting examples of methods of transforming plants, plant parts and plant cells include Agrobacterium-mediated transformation (De Blaere et al. (1987) Meth. Enzymol. 153:277-293), particle bombardment-mediated transformation (Klein et al. (1987) Nature 327:70-73; as well as U.S. Pat. No. 4,945,050), calcium-phosphate-mediated transformation, cyclodextrin-mediated transformation, electroporation, liposome-mediated transformation, nanoparticle-mediated transformation, polymer-mediated transformation, virus-mediated transduction, and the like. See, e.g., Bacchetti & Graham (1977) Proc. Natl. Acad. Sci. USA 74:1590-1594; Bertram (2006) Current Pharm. Biotechnol. 7:277-228; Fischer et al. (2002) Bioconjugate Chem. 13:1124-1133; Graham & van der Eb (1973) Virology 52:456-467; Menuel et al. (2008) Bioconjugate Chem. 19:2357-2362; Soltani et al., “Agrobacterium-mediated transformation of non-plant organisms” 649-675 In: Agrobacterium: From Biology to Biotechnology (Tzfira & Citovsky eds., Springer 2008); and Tsukakoshi et al. (1984) Appl. Physics B-Photophys. Laser Chem. 35:135-140. Methods of plant transformation are described in more detail below.

A nucleotide sequence therefore can be introduced into the plant, plant part or plant cell in any number of ways that are well known in the art. The methods of the invention do not depend on a particular method for introducing one or more nucleotide sequences into a plant, only that they gain access to the interior of at least one cell of the plant. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, in separate transformation events, or, for example, in plants, as part of a breeding protocol.

In addition to expression cassettes, numerous plant transformation vectors are well known to one of skill in the art, and the nucleotide sequences described herein can be used in connection with any such vectors. The selection of a vector will depend upon the preferred transformation technique and the target species for transformation. For certain target species, different antibiotic or herbicide selection markers may be preferred as discussed elsewhere herein.

For example, Ti plasmid vectors have been used to deliver foreign DNA, as well as direct DNA uptake, liposomes, electroporation, microinjection and microprojectiles. In addition, bacteria from the genus Agrobacterium can be used to transform plant cells. Below are descriptions of representative methods for transforming both monocotyledonous (monocots) and dicotyledonous (dicots) plants, as well as representative plastid transformation methods.

Many vectors are available for transformation using Agrobacterium tumefaciens. These typically carry at least one T-DNA border sequence and include vectors such as pBIN19 (Bevan (1984) Nucleic Acids Res. 12: 8711-8721). U.S. Patent Application Publication No. 2006/0260011 describes methods for constructing vectors useful in Agrobacterium-mediated transformation.

Transformation without the use of A. tumefaciens circumvents the requirement for T-DNA sequences in the chosen transformation vector and consequently vectors lacking these sequences also can be used. Transformation methods that do not rely on Agrobacterium include transformation via microinjection, particle bombardment, and protoplast uptake (e.g., PEG and electroporation). As noted above, the choice of vector will depend largely on the preferred selection for the species being transformed.

Methods of transforming monocots are well known in the art and include direct nucleic acid transfer into protoplasts using PEG- or electroporation-mediated methods, as well as particle bombardment. Transformation can be undertaken with a single nucleic acid molecule or multiple nucleic acid molecules (e.g., co-transformation) and both of these methods are suitable for use herein. Co-transformation may have the advantage of avoiding complete vector construction and of generating transgenic plants with unlinked loci for the nucleic acid of interest and the selectable marker, enabling the removal of the selectable marker in subsequent generations, should this be regarded as desirable.

EP Patent Application Nos. 0 292 435 and 0 392 225, as well as Int'l Patent Application Publication No. WO 93/07278, describe methods for preparing callus and protoplasts from an elite inbred line of maize, transformation of protoplasts using PEG or electroporation, and the regeneration of maize plants from transformed protoplasts. See, Fromm et al. (1990) Bio/Technology 8:833-844; and Gordon-Kamm et al. (1990) Plant Cell 2:603-618, which describe methods for transforming A188-derived maize lines using particle bombardment. Furthermore, Int'l Patent Application Publication No. WO 93/07278 and Koziel et al. (1993) Biotechnol. 11:194-200 describe methods for transforming elite inbred lines of maize by particle bombardment. These methods use immature maize embryos of 1.5-2.5 mm length excised from a maize ear 14-15 days after pollination and a PDS-1000He Biolistics device for bombardment. In each case, the transformed cells can be regenerated to whole plants using methods well known in the art.

Methods of transforming dicots also are well known in the art and include Agrobacterium-mediated methods as well as methods that do not require Agrobacterium. Non-Agrobacterium-mediated nucleic acid delivery methods involve the uptake of exogenous genetic material directly by protoplasts or cells. This method can be accomplished by, for example, PEG- or electroporation-mediated uptake, particle bombardment-mediated delivery or microinjection. Examples of these methods are described in, for example, Klein et al. (1987) Nature 327:70-73; Paszkowski et al. (1984) EMBO J. 3:2717-2722; Potrykus et al. (1985) Mol. Gen. Genet. 199:169-177; and Reich et al. (1986) Biotechnology 4:1001-1004. In each case, the transformed cells can be regenerated to whole plants using methods well known in the art.

Agrobacterium-mediated transformation is a preferred method for transforming dicots because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hofgen & Willmitzer (1988) Nucleic Acids Res. 16:9877).

Transformation of the target plant species by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the cells under conditions effective to penetrate the outer surface of the cell and afford incorporation within the interior thereof. When inert particles are utilized, the vector can be introduced into the cell by coating the particles with the vector containing a nucleotide sequence of interest. Alternatively, the target cell can be surrounded by the vector so that the vector is carried into the cell by the wake of the particle. Biologically active particles (e.g., dried yeast cells, dried bacterium or a bacteriophage, each containing one or more nucleic acids sought to be introduced) also can be propelled into plant tissue.

The plants obtained via transformation with a nucleotide sequence of interest can be any of a wide variety of plant species, including monocots and dicots. In particular embodiments, the plants used in the method of the invention are selected from the list of agronomically important target crops set forth supra. The expression of a nucleotide sequence of interest in combination with other characteristics important for production and quality can be incorporated into plant lines through breeding. Breeding approaches and methods are known in the art. See, e.g., Welsh, Fundamentals of Plant Genetics and Breeding (John Wiley & Sons 1981); Crop Breeding (Wood ed., American Society of Agronomy 1983); Mayo, The Theory of Plant Breeding, 2^{nd} ed. (Clarendon Press 1987); Singh, Breeding for Resistance to Diseases and Insect Pests (Springer-Verlag 1986); and Wricke & Weber, Quantitative Genetics and Selection Plant Breeding (Walter de Gruyter and Co. 1986).

Whole plants may be regenerated from transgenic cells by methods well known in the art. See, e.g., Fromm et al. (1990), supra; and Gordon-Kamm et al. (1990), supra.

Likewise, the genetic properties engineered into the transgenic seeds and plants described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

The dual activity enzymes of the present invention also can be incorporated into or maintained in plant lines through breeding or through common genetic engineering technologies. See, id. The relevant breeding methods include, but are not limited to, aneuploid techniques, backcross breeding, dihaploid inbreeding, hybridization, inbreeding, interspecific hybridization, multi-line breeding, variety blend, etc. Hybridization techniques also include the sterilization of plants to yield male or female sterile plants by biochemical, chemical, genetic (including transgenic) or mechanical means.

Transgenic organisms of the invention also include non-plant cells and non-plant organisms transformed with a nucleic acid construct encoding the dual activity enzyme, variant or fragment thereof having xylanase and cellulase activity. As used herein, “cell” means a cell in which the expression cassette (including a vector comprising the expression cassette) can be propagated and expressed. A cell also includes any progeny of the subject cell or its derivatives. It is understood that all progeny may not be identical to the parental cell since there may be mutations that occur during replication; however, such progeny are included within “cell.” Examples of cells for use herein include, but are not limited to, prokaryotic cells such as bacterial cells, and eukaryotic cells such as fungal (e.g., yeast cells), insect (e.g., Drosophila cells) and mammalian cells. In some particular embodiments, examples of non-plant cells include, but are not limited to, ethanologenic organisms discussed above, and yeast, especially Aureobasidium, Cryptococcus, Kluyveromyces, Rhodotorula, Saccharomyces and Sporobolomyces.

Briefly, methods of expressing a nucleotide sequence in a non-plant cell or non-plant organism includes transforming the cell or organism with, for example, a nucleic acid molecule comprising a coding sequence for the dual activity enzyme, or biologically active variant or fragment thereof, operably linked to one or more regulatory sequences. In some embodiments, the coding sequence for the dual activity enzyme is the nucleotide sequence of SEQ ID NO:1 and/or SEQ ID NO:3, or an active fragment or variant of SEQ ID NO:1 and/or SEQ ID NO:3. In other embodiments, the coding sequence for the dual activity enzyme is any nucleotide sequence encoding the amino acid sequence of SEQ ID NO:2 and/or SEQ ID NO:4, or an active fragment or variant of SEQ ID NO:2 and/or SEQ ID NO:4. Transformed cells are grown under conditions where the nucleotide sequence is expressed.

Methods for constructing expression cassettes are described above. Once constructed, the expression cassette can be incorporated into a vector to assist in introducing the expression cassette into the cell.

Methods of introducing nucleic acid constructs such as expression cassettes and vectors into non-plant cells are well known in the art and will vary depending upon the cell selected. See, e.g., Davis et al., Basic Methods in Molecular Biology (Elsevier Press 1986). Examples of methods for introducing nucleic acid constructs such as expression cassettes or vectors into cells include, but are not limited to, Agrobacterium-mediated transformation, calcium-phosphate-mediated transformation, cyclodextrin-mediated transformation, electroporation, liposome-mediated transformation, nanoparticle-mediated transformation, particle bombardment, polymer-mediated transformation, and virus-mediated transduction. See, e.g., Bacchetti & Graham (1977) Proc. Natl. Acad. Sci. USA 74:1590-1594; Bertram (2006) Current Pharm. Biotechnol. 7:277-228; Fischer et al. (2002) Bioconjugate Chem. 13:1124-1133; Graham & van der Eb (1973) Virology 52:456-467; Menuel et al. (2008) Bioconjugate Chem. 19:2357-2362; Soltani et al., “Agrobacterium-mediated transformation of non-plant organisms” 649-675 In: Agrobacterium: From Biology to Biotechnology (Tzfira & Citovsky eds., Springer 2008); and Tsukakoshi et al. (1984) Appl. Physics B-Photophys. Laser Chem. 35:135-140.

As above, the non-plant cell or non-plant organism can be transiently transformed or stably transformed.

Once the dual activity enzyme, active variant or fragment thereof is expressed by the cell or organism, it can be purified from the cell or organism for use in the compositions described herein. Alternatively, the transformed cell or organism expressing the enzymes of the invention can be used directly without purification of the enzymes. For example, transformed plant(s) expressing the dual function enzyme can be used directly. Thus, in some embodiments, plants expressing the dual function enzyme can be added to, for example, other feedstock, all of which can be chopped up or steam exploded. Ammonia based liquid can then be added to the chopped up/exploded feedstock to break down the plant material into small carbohydrates.

Thus, the isolated enzymes and/or the transgenic cells and organisms can be used in lignocellulose bioprocessing. In this manner, the compositions can be formulated at a high pH. Thus, in some embodiments, the pH of the compositions is about pH 8.5 or higher. Accordingly, in other embodiments of the invention, the pH of the compositions can be at least about 8.5, about 9.0, about 9.5, about 10, about 10.5, about 11, about 11.5, about 12 and higher. In some particular embodiments, the pH of the compositions is about 12. Other components can be included within the composition including, but not limited to, other bioprocessing enzymes or chemicals as well as stabilizers.

Methods

Methods of the invention include contacting lignocellulosic material with an enzyme composition as described herein to hydrolyze the cellulose, hemicellulose and lignin polymers (i.e., lignocellulosic plant material) therein into monomers for subsequent use in various feed, food/beverage and technical industries. In particular, due to the stability of the dual activity enzymes at high temperature and high pH, the methods of the present invention find use in high temperature and high pH applications. Thus, an advantage of the present invention is that enzyme hydrolysis of the lignocellulosic material can be carried out at the same time as an alkaline treatment of the lignocellulosic material (e.g., the xylanase enzyme hydrolysis can be carried out under alkaline conditions (i.e., in an alkaline composition)). The cellulase activity of the dual activity enzyme, which is retained at both high pH and high temperature, can later be activated by reducing the pH.

Briefly, bioconversion of lignocellulosic materials to useful, higher value products can involve multi-step processes including: (1) pretreatment (e.g., biological, chemical or mechanical) of the lignocellulosic material, and (2) hydrolysis of cellulose, hemicellulose and/or lignin polymers to produce simpler, yet readily metabolizable, molecules (e.g., hexose or pentose sugars) by contacting the lignocellulosic material with a dual activity enzyme of the present invention (e.g., SEQ ID NO:2 and/or SEQ ID NO:4), or a biologically active variant or fragment thereof. Additional steps optionally can be performed, such as may be required in the feed, food/beverage and technical industries. Such additional steps can include: (3) bio-utilization of the molecules to support microbial growth or to produce chemical products, and (4) separation and purification of the chemical products.

Lignocellulose can be found, for example, in the stems, leaves, hulls, husks and cobs of non-woody plants, or leaves, branches and wood of woody plants such as trees. Lignocellulosic material also can be obtained from lignocellulosic waste products, such as plant residue and paper waste. Examples of plant residues include, but are not limited to, stems, leaves, hulls, husks, cobs and the like, as well as wood, wood chips, wood pulp and sawdust. Examples of paper waste include, but are not limited to, discarded photocopy paper, computer printer paper, notebook paper, notepad paper, typewriter paper and the like, as well as discarded newspapers, magazines, cardboard and paper-based packaging materials. Lignocellulosic material also can be from agricultural residues, forestry residues, herbaceous material, municipal solid wastes, and pulp and paper mill residues.

Thus, in one embodiment of the present invention a method of processing lignocellulosic material is provided, the method comprising contacting the lignocellulosic material with an alkaline composition at a pH of at least about 10 and an enzyme comprising the amino acid sequence of SEQ ID NO:2 and/or SEQ ID NO:4, whereby the lignocellulosic material is processed. In some embodiments of the present invention, a method is provided wherein the lignocellulosic material is provided by a plant, plant part and/or plant cell transformed with a nucleotide sequence encoding the amino acid sequence of SEQ ID NO:2 and/or SEQ ID NO:4, thereby providing the enzyme comprising the amino acid sequence of SEQ ID NO:2 and/or SEQ ID NO:4 in the lignocellulosic material. Thus, in some embodiments, a method of processing lignocellulosic material is provided, the method comprising contacting the lignocellulosic material with an alkaline composition having a pH of at least about 10, wherein the lignocellulosic material comprises a plant, plant part or plant cell transformed with an enzyme having the amino acid sequence of SEQ ID NO:2 and/or SEQ ID NO: 4, whereby the lignocellulosic material is processed.

As used herein an “alkaline composition” comprises any alkaline condition known by those of skill in the art to be used in alkaline treatment or pretreatment of lignocellulosic materials. Non-limiting examples of alkaline compositions or alkaline pretreatments include sodium hydroxide, potassium hydroxide, calcium hydroxide, ammonium hydroxide, ammonia, and the like, or any combination thereof. In some embodiments of the invention, the pH of an alkaline composition of the present invention is at least about 10. In other embodiments, the pH of the alkaline composition is in a range from about 10 to about 12. In still other embodiments, the pH of the alkaline composition is about 11. In further embodiments, the pH of the alkaline composition is about 12.

In some embodiments, the lignocellulosic material is obtained from a plant, plant part or plant cell that is transformed with a nucleotide sequence encoding an active variant or active fragment of the amino acid sequence of SEQ ID NO:2 and/or SEQ ID NO:4. In some embodiments, the lignocellulosic material further comprises lignocellulosic material from a plant, plant part or plant cell not transformed with an enzyme comprising the amino acid sequence of SEQ ID NO:2 and/or SEQ ID NO:4. Lignocellulosic material from a plant, plant part or plant cell not transformed with an enzyme comprising the amino acid sequence of SEQ ID NO:2 and/or SEQ ID NO:4 can be lignocellulosic material from a transgenic plant, plant part or plant cell or a non-transgenic plant, plant part or plant cell. Thus, in some embodiments, the lignocellulosic material from a plant, plant part or plant cell not transformed with the nucleotide sequence encoding the amino acid sequence of SEQ ID NO:2 and/or SEQ ID NO:4 can be transformed with a nucleotide sequence encoding an enzyme other than the enzyme comprising the amino acid sequence of SEQ ID NO:2 and/or SEQ ID NO:4.

The methods of the present invention can further comprise contacting the lignocellulosic material with at least one other enzyme in addition to the dual activity enzyme of the invention. Thus, in some embodiments, the methods of the invention further comprise contacting the lignocellulosic material with at least one additional enzyme selected from the group consisting of a cellulase, hemicellulase, ligninase, pectinase, protease, and any combination thereof.

In some particular embodiments, the cellulase is provided by an enzyme having the amino acid sequence of SEQ ID NO:2 and/or SEQ ID NO:4. In other embodiments, the cellulase is selected from the group consisting of a mannan endo-1,4-β-mannosidase, 1,3-β-D-glucan glucanohydrolase, 1,3-β-glucan glucohydrolase, 1,3-1,4-β-D-glucan glucanohydrolase, 1,6-β-D-glucan glucanohydrolase, and any combination thereof.

In some embodiments, the hemicellulase is an α-L-arabinofuranosidase, α-glucuronidase, acetyl mannan esterase, acetyl xylan esterase, α-galactosidase, glucosidase, β-1,4-xylosidase, endo-galactanase, endo-β-1,4-mannanase, endo-α-1,5-arabinanase, exo-β-1,4-mannosidase, exo-β-1,4-xylosidase, feruloyl esterase, ferulic acid esterase, p-cumaric acid esterase, glucuronoxylan xylanohydrolase, xyloglucan endotransglycosylase, or any combination thereof.

In other embodiments, the ligninase is a diarylpropane peroxidase, glucose oxidase, glyoxal oxidase, lignin peroxidase, manganese peroxidase, methanol oxidase, methanol oxidoreductase, phenol oxidase, phenol peroxidase, veratryl alcohol oxidase, or any combination thereof.

In still other embodiments, pectinase is a pectolyase, pectozyme, polygalacturonase, or any combination thereof.

Non-limiting examples of a protease includes an asclepain, bromelain, caricain, chymopapain, collagenase, glycyl endopeptidase, pepsin, pronase, subtilisin, thermolysin, or any combination thereof.

Additional embodiments of the present invention provide contacting the lignocellulosic material with at least one enzyme selected from the group consisting of an amylase, catalase, cutinase, glucanase, glucoamylase, glucose isomerase, lipase, phytase, pullulanase, xylose isomerase, and any combination thereof.

In some embodiments of the present invention, the additional enzyme can be provided by the lignocellulosic material obtained from a plant, plant part or plant cell that is transformed with an active variant or active fragment of the amino acid sequence of SEQ ID NO:2 and/or SEQ ID NO:4, which is further transformed with one or more than one nucleic acid molecule encoding an additional enzyme as described above.

In further embodiments, the method of the present invention comprises contacting (e.g., fermenting) the lignocellulosic material with an ethanologenic bacteria or yeast or a combination thereof. In some embodiments, the contacting of the lignocellulosic material with an ethanologenic bacteria or yeast or a combination thereof is at a pH of about 6 to about 8.

While the lignocellulosic material can be used “as is,” it also can be pretreated, as mentioned above. Any pretreatment known in the art can be used with the methods of the present invention. Thus, in some embodiments, the present invention provides methods of pretreating the lignocellulosic material with a biological pretreatment, a chemical pretreatment, a physical pretreatment, or any combination thereof.

As used herein, “pretreating,” “pretreatment,” and other grammatical variations thereof, refer to exposing lignocellulosic material to a treatment prior to contacting the lignocellulosic material with the alkaline composition. Pretreatment of the lignocellulosic material opens up the lignocellulosic matrix and makes the cellulosic fibers more available for further processing. Specifically, through pretreatment the lignocellulosic material is made more available to further processing by enzyme digestion.

Examples of biological pretreatment include, but are not limited to, pretreating with lignin-solubilizing microorganisms such as Clostridium spp. or Streptomyces spp.

Non-limiting examples of chemical pretreatment include alkaline treatment (e.g., lime, sodium hydroxide, sodium carbonate, ammonia treatment, ammonium sulfite, and the like), acid treatment (e.g., dilute acid treatment, carbonic acid, hydrochloric, hydrofluoric, nitric, peracetic acid, phosphoric acid, sulfur dioxide, sulfuric acid, and the like), organic solvent treatment, oxidant treatment (e.g., ozone treatment, wet oxidation, hydrogen peroxide, and the like), carbon dioxide treatment, pH-controlled hydrothermolysis, and/or any combination thereof.

Examples of physical pretreatment include, but are not limited to, various types of hydrothermolysis, irradiation, milling, steaming/steam explosion, sonicating (e.g., ultra-sonicating), expansion, extrusion, and/or any combination thereof. See generally, Hsu, “Pretreatment of biomass” 179-212 In: Handbook on Bioethanol—Production and Utilization (Wyman ed., Taylor & Francis 1996); Ghosh & Singh (1993) Adv. Appl. Microbiol. 39:295-333; McMillan, “Pretreating lignocellulosic biomass: a review” Chapter 15 In: Enzymatic Conversion of Biomass for Fuels Production (Himmel et al. eds., American Chemical Society 1994); Gong et al. (1999) Adv. Biochem. Eng./Biotechnol. 65:207-241; Olsson & Hahn-Hagerdal (1996) Enz. Microb. Tech. 18:312-331; and Vallander & Eriksson (1990) Adv. Biochem. Eng./Biotechnol. 42:63-95; as well as U.S. Pat. Nos. 5,733,741 and 6,333,181.

In some additional aspects of the present invention, the method further comprises contacting the lignocellulosic material with at least one agent selected from the group consisting of chlorine, detergent, hypochlorite, hydrogen peroxide, oxalic acid, peracid, pH-regulating agent, trisodium phosphate, sodium chlorite, sodium nitrate, surfactant, urea, water, and any combination thereof. The contacting of the lignocellulosic material with the at least one agent can occur prior to, concurrently with, or after contacting the lignocellulosic material transformed with the dual active enzyme of the invention with an alkaline composition.

Thus, as described above, some embodiments of the present invention provide methods comprising contacting lignocellulosic material having cellulose, hemicellulose and/or lignin and transformed with a dual activity enzyme with an alkaline composition at a pH of at least about 10, thereby hydrolyzing the lignocellulosic material to simpler, more readily utilizable molecules. As used herein, “contacting” means bringing together the lignocellulosic material, the enzyme (or variant or fragment thereof) and the alkaline composition or alkaline conditions thereby facilitating intermolecular interactions necessary to cause the alkaline composition and the enzyme (or active variant or fragment thereof) to hydrolyze or act upon the lignocellulosic material. Depending upon the source of the enzyme, and/or active variant or fragment thereof, contacting can occur in vitro, ex vivo or in vivo. Thus, in some embodiments, contacting comprises grinding, crushing, mashing, squeezing, pulverizing, sonicating, chopping, etc., the plant, plant part or plant cell expressing a nucleotide sequence encoding an enzyme of the present invention (e.g., an enzyme comprising the amino acid sequence of SEQ ID NO:2 and/or SEQ ID NO:4) in the presence of an alkaline composition, thereby bringing the expressed enzyme into contact with the lignocellulosic material of the plant, plant part and/or plant cell under alkaline conditions. The amount of enzyme to be used with the methods of the present invention is as described above. Thus, in some embodiments, the enzyme is present in an amount from about 0.01 units to about 1000 units. In other embodiments, enzyme is present in an amount of about 500 units.

The contacting time depends upon factors such as the result desired, the type of lignocellulosic material used, the amount of lignocellulosic material used, the amount of enzyme used, the specific activity of the enzyme composition, the temperature, the pH, and the like. This step, however, can be carried out for a time sufficient to decrease the particle size and degree of polymerization of the cellulose and hemicellulose and to delignify the lignocellulosic material.

Thus, in some embodiments, the lignocellulosic material can be contacted with the enzyme, and/or active variant or fragment thereof, and the alkaline composition for a period of time in a range from about 5 min to about 48 hours. In other embodiments, the lignocellulosic material comprising the dual activity enzyme can be contacted with the alkaline composition for about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, or more. In some embodiments, the contacting is for about 12 hours to about 24 hours. In additional embodiments, the contacting is for about 10 to 12 hours. In other embodiments, the contacting is for about 30 minutes to about 6 hours. In further embodiments, the contacting is for about 6 hours.

As noted above, the contacting is carried out at a temperature and pH that enhances the activity of the enzyme in vitro. In some embodiments, the temperature can be in a range from about 10° C. to about 100° C. In other embodiments, the temperature can be in a range from about 65° C. to 95° C. Thus, in some embodiments, the contacting is carried out at a temperature of about 10° C., 15° C., 20° C., 25° C., 30° C., 35° C., 40° C., 45° C., 50° C., 60° C., 65° C., 70° C., 75° C., 80° C., 85° C., 90° C., 95° C., and the like. In some particular embodiments, the contacting is carried out at a temperature of 95° C.

The pH for contacting of the lignocellulosic material transformed with the dual activity enzyme with the alkaline composition can be from about 10 to about 12. It is a characteristic of the dual activity enzymes of the present invention that they can be active over a wide pH-range as well as having high activity at pH 6-12, which is the pH required for many processes in the feed, food/beverage and technical industries such as, for example, in the storage towers used by the pulp mills. Because the enzyme is active over a wide pH-range, the pH can be varied during the methods. As noted above, the enzyme has both xylanase and cellulase activity at about pH 6, and retains both activities even up to about pH 12. Thus, the pH can begin at about 6, in which both xylanase and cellulase activity is present, and then can be steadily (i.e., gradually) changed to a more basic pH such as 12, in which only the xylanase is active; however, the cellulase is not destroyed and instead retains the ability to become active once the pH is lowered to a pH in which the cellulase is active. Alternatively, the pH for the contacting can begin at about 10 to about 12, and then over time be changed steadily to a more acidic pH such as pH 6. As used herein, “over time” refers to a gradual and steady change in the pH from the starting pH to the ending pH over a period of about 3 minutes to about 2 hours. Thus, for example, the contacting of the lignocellulosic material with the enzyme, and/or active variant or fragment thereof, and the alkaline composition (e.g., pH 10-12) can be for a period of time in a range from about 10 hours to about 12 hours at the end of which the pH can be steadily lowered to 6 over a period of time of 3 minutes to about 2 hours.

Thus, in some embodiments of the present invention, the pH is reduced over time from a pH in a range from about 10 to about 12 to a pH in a range from about 6 to about 8. In some particular embodiments, the pH is reduced over time from a pH of about 12 to a pH of about 6. In other embodiments, the pH for contacting begins at about 11 and is reduced over time to a pH of about 6. In still other embodiments, the pH for contacting starts at about 10 and is reduced over time to a pH of about 6 to about 8. In additional embodiments, the pH for contacting starts at about 10 and is reduced over time to a pH of about 6. In further embodiments, the pH is reduced over time from a pH of about 12 to a pH of about 7. In other embodiments, the pH for contacting begins at about 11 and is reduced over time to a pH of about 7. In still other embodiments, the pH for contacting starts at about 10 and is reduced over time to a pH of about 7. Other embodiments of the present invention provide methods wherein the pH for contacting starts at about 12 and is reduced over time to a pH of about 8. In still other embodiments, the pH for contacting starts at a pH of about 11 and is reduced over time to a pH of about 8. In further embodiments, the pH for contacting starts at about pH 10 and is reduced over time to a pH of about 8.

The enzyme compositions or modified organisms can be used in various methods. The dual activity enzymes have the advantage that they can be used at high temperatures and high pHs. In one embodiment, the methods can be used in the feed industry to make animal feeds. The methods provide for hydrolyzing xylans and cellulases in a feed prior to or during consumption by an animal comprising the following steps: (a) obtaining a feed material comprising lignocellulosic material such as cellulose, hemicellulose and lignin; and (b) contacting an enzyme composition comprising a dual activity enzyme (for example, SEQ ID NO:2 and/or SEQ ID NO:4), or a variant or fragment thereof having xylanase and/or cellulase activity, to the feed material in a sufficient amount and for a sufficient time period to cause hydrolysis of xylans and celluloses, thereby hydrolyzing the xylans and celluloses in the feed prior to or during consumption by the animal. The methods also can include (c) administering the feed material to the animal, wherein after consumption, the xylanase and/or cellulase activity causes hydrolysis of xylans and/or celluloses of the feed in the digestive tract of the animal. The feed can be, for example, a cereal, corn, grain, and the like.

In some embodiments of the present invention, the solid by-products of the production of ethanol and other biofuels from the lignocellulosic material can be used for animal feed. Thus, once the lignocellulosic material has been processed and ethanol has been extracted, the remaining solids and liquids can be separated. The separated solids can be used as animal feed. Alternatively, the separated solids can be used as fertilizer.

In another embodiment, the methods can be used in the food/beverage industry to process fruit or vegetable materials. The methods provide for hydrolyzing xylans and celluloses in a food or beverage prior to consumption by a human comprising the following steps: (a) providing a fruit or vegetable material comprising lignocellulosic material such as cellulose, hemicellulose and lignin such as fruit or vegetable material; and (b) contacting an enzyme composition comprising a dual activity enzyme (for example, SEQ ID NO:2 and/or SEQ ID NO:4), or a variant or fragment thereof having xylanase and/or cellulase activity, to the fruit or vegetable material in a sufficient amount and for a sufficient time period to cause hydrolysis of xylans and celluloses, thereby processing the fruit or vegetable material. The food can be, for example, such a fruit or vegetable juice, beer, wine, syrup, puree or extract and the like.

In another embodiment, the methods can be used in the technical industry to treat lignocellulosic plant material. For example, the methods can be used to in the paper making industry to treat paper pulp. Thus, the methods provide for hydrolyzing xylans and celluloses in lignocellulosic plant materials comprising the following steps: (a) providing a lignocellulosic plant material such as crop, plant and tree biomass comprising cellulose, hemicellulose and lignin; (b) contacting an enzyme composition comprising a dual activity enzyme (for example, SEQ ID NO:2 and/or SEQ ID NO:4), or a variant or fragment thereof having xylanase and cellulase activity, with lignocellulosic plant material in a sufficient amount and for a sufficient time period to cause hydrolysis of xylans and celluloses, thereby processing the lignocellulosic plant material. The methods also can include prior to step (b), when bleaching lignocellulosic plant material (e.g., pulpy materials), exposing lignocellulosic material to a chlorine dioxide bleaching stage to produce a partially bleached lignocellulosic material (e.g., a partially bleached pulp); (b) contacting the partially bleached lignocellulosic material with the enzyme composition comprising the enzyme, variant or fragment thereof having xylanase and/or cellulase activity; and (c) exposing the treated lignocellulosic material to a second chlorine dioxide bleaching without an alkaline extraction stage.

In another embodiment, the methods can be used in the technical industry to produce ethanol. The methods provide for hydrolyzing xylans and celluloses in non-woody or woody plant materials comprising the following steps: (a) providing a non-woody or woody plant material such as crop, plant or tree biomass comprising cellulose, hemicellulose and lignin; (b) contacting an enzyme composition comprising a dual activity enzyme (for example, SEQ ID NO:2 and/or SEQ ID NO:4), or a variant or fragment thereof having xylanase and/or cellulase activity, with the non-woody or woody plant material in a sufficient amount and for a sufficient time period to cause hydrolysis of xylans and celluloses, thereby processing the non-woody or woody plant material for subsequent ethanol production.

In another embodiment, the methods can be used to make biofuels. The methods provide for hydrolyzing xylans and celluloses in non-woody or woody plant materials comprising the following steps: (a) providing a non-woody or woody plant material such as crop, plant or tree biomass comprising cellulose, hemicellulose and lignin; (b) contacting an enzyme composition comprising a dual activity enzyme (for example, SEQ ID NO:2 and/or SEQ ID NO:4), or a variant or fragment thereof having xylanase and/or cellulase activity, with the non-woody or woody plant material in a sufficient amount and for a sufficient time period to cause hydrolysis of xylans and celluloses, thereby processing the non-woody or woody plant material for subsequent biofuel production.

In further embodiments, the present invention provides a method for processing lignocellulosic material for production of fermentable sugars or for ethanol production, the method comprising: contacting lignocellulosic material with an alkaline composition at a pH of at least about 10, wherein the lignocellulosic material comprises a plant, plant part or plant cell transformed with a nucleotide sequence encoding a dual activity enzyme (for example, an enzyme having the amino acid sequence of SEQ ID NO:2 and/or SEQ ID NO:4), whereby the lignocellulosic material is processed for production of fermentable sugars or for ethanol production.

In a still further embodiment, the present invention provides a method for processing lignocellulosic material for biofuel production, the method comprising: contacting lignocellulosic material with an alkaline composition at a pH of at least about 10, wherein the lignocellulosic material comprises a plant, plant part or plant cell transformed with a nucleotide sequence encoding a dual activity enzyme (for example, an enzyme having the amino acid sequence of SEQ ID NO:2 and/or SEQ ID NO:4), whereby the lignocellulosic material is processed for biofuel production.

The present invention is more particularly described in the following examples that are intended as illustrative only since numerous modifications and variations therein will be apparent to those skilled in the art.

### EXPERIMENTAL

### Example 1

### Xylanase Having Cellulase Activity

This example shows that the xylanase from D. thermophilum (Gibbs et al. (1995), supra; referred to hereinbelow as DtXynA) has cellulase activity.

Recombinant Enzyme Production:

An E. coli codon-optimized nucleotide sequence for DtXynA (SEQ ID NO:1) was cloned into a Novagen® pET24a Bacterial Expression Vector (EMD Chemicals, Inc.; Gibbstown, N.J.) using the NdeI/HindIII restriction sites and then expressed in E. coli MC1061DE3 competent cells.

A culture of cells in 2 ml of LB medium containing 25 μg/ml kanamycin was incubated overnight. The culture then was used to inoculate (1%) a 50 ml culture in a 250 ml Erlenmeyer flask. The culture was grown at 37° C. under shaking (180 rpm) until the OD₆₀₀ₙₘ was 0.4.

To induce DtXynA production, cells were treated with 250 μM isopropyl β-D-1-thiogalactopyranoside (IPTG; Carbosynth; Compton, Berkshire, United Kingdom) and incubated at 20° C. for 16 hours. Following this incubation, the OD₆₀₀ₙₘ=2.5.

DtXynA was purified to about 50% using standard techniques for protein purification from recombinant bacteria.

Cellulase Assay:

An assay for cellulase activity was performed using 0.25% carboxymethyl cellulose (CMC) as a substrate. The positive control was Trichoderma longibrachiatum endoglucanase II (EGII; Megazyme Int'l Ireland Ltd.), which is known to have cellulase activity. The negative control was a protein extract from a recombinant E. coli comprising the pET24a vector which contains no insert.

Briefly, 0, 10, 20 or 50 μg enzyme/mg CMC was incubated for 0, 4, 8 or 24 hours in 50 mM sodium acetate, pH 5.3 at 60° C. The increased incubation time was to account for the different nature of EGII, which typically has a slower cellulase activity than the cellulase of the dual function xylanases/cellulases of the present invention.

Cellulase activity was measured using a 3,5-dinitrosalicyclic acid (DNS) assay as described in Miller (1959), supra, which colorimetrically measures reducing ends of the cleaved sugar polymers at 540 nm. For each time point, known standards of 0, 20, 40, 60, 80 and 100 mM xylose were used to generate a standard curve. The amount (mM) of reducing ends released by the enzyme was calculated by subtracting out background activity (A540 at 0 hours) and plotting the A540 on the standard curve.

Results:

As shown in Table 1, DtXynA possesses cellulase activity that was dose-dependent and increased for each dose over the 24 hour period. In contrast, the cellulase activity of EGII appeared saturated and was stable for each dose over the 24 hour period. As expected, cellulase activity of the empty vector control was negligible.

TABLE 1
Cellulase activity of DtXynA and EGII.

| Treatment | 4 hours | 8 hours | 24 hours |
|---|---|---|---|
| DtXynA, 10 ug | 8.97 | 15.01 | 26.97 |
| DtXynA, 20 ug | 17.56 | 23.60 | 36.29 |
| DtXynA, 50 ug | 26.64 | 30.16 | 43.88 |
| EGII, 10 ug | 23.59 | 24.09 | 26.14 |
| EGII, 20 ug | 20.32 | 22.23 | 23.13 |
| EGII, 50 ug | 16.14 | 19.97 | 18.55 |
| Empty, 10 ug | 2.57 | 3.59 | 1.45 |
| Empty, 20 ug | 3.96 | 4.55 | 4.57 |
| Empty, 50 ug | 2.68 | 3.80 | 1.92 |

Data expressed as mM reducing sugar ends released following 60° C. incubation with varying amounts of enzyme. Data shown are the average of three replicates; standard error is less than 0.01% and is not shown.

### Example 2

### Optimal pH of Cellulase Activity of DtXynA

This example shows that DtXynA has optimal cellulase activity at a pH between pH 5 and pH 7.

Methods:

Recombinant Enzyme Production:

cells expressing recombinant DtXynA were produced as described above in Example 1.

Optimal pH Assay:

Cells were centrifuged at 8000×g for 5 minutes. The supernatant was discarded, and cells were re-suspended at OD₆₀₀ₙₘ=0.02 in a 0.1 M sodium citrate buffer (pH 3.0 to 6.0) or a 0.1 M sodium phosphate buffer (pH 6.0 to 9.0). 50 μl of re-suspended cells a pH of 3, 4, 5, 6, 7, 8 or 9 was added to 50 μl of 4 mM p-nitrophenyl β-D-cellobioside (Sigma; St. Louis, Mo.) as a substrate for the enzyme. The cells were incubated for 1 hour at 70° C. After the 1 hour incubation, 100 μl of 0.2 M Na₂CO₃ was added to the cells, and the released p-nitrophenol was measured at 414 nm.

Results:

As shown in Table 2, DtXynA's cellulase activity was optimal at pH 6, with about 50% activity at pH 5 and 7.

TABLE 2
Optimal pH of cellulase
activity of DtXynA.

| pH | ΔOD_{414 nm} |
|---|---|
| 3 | −0.0022 |
| 4 | −0.0004 |
| 5 | 0.2991 |
| 6 | 0.4875 |
| 7 | 0.2209 |
| 8 | 0.0437 |
| 9 | −0.0114 |

### Example 3

### Optimal Temperature of Cellulase Activity of DtXynA

This example shows that the cellulase activity of DtXynA begins around 60° C. and reaches its optimal activity at 95° C. at pH 6.0.

Methods:

Recombinant Enzyme Production:

cells expressing recombinant DtXynA were produced as described above in Example 1.

Temperature Assay:

cells were centrifuged at 8000×g for 5 minutes. The supernatant was discarded, and cells were re-suspended at OD₆₀₀ₙₘ=0.02 in a 0.1 M sodium phosphate buffer at pH 6.0. 50 μl of re-suspended cells was added to 50 μl of 4 mM p-nitrophenyl β-D-cellobioside as a substrate for the enzyme. Cells were incubated for 1 hour at temperatures between 37° C. and 95° C. After the 1 hour incubation, 100 μl of 0.2 M Na₂CO₃ was added to the cells, and the released p-nitrophenol was measured at 414 nm.

Results:

As shown in Table 3, DtXynA's cellulase activity was present at 45° C. to 95° C. and was optimal at 95° C.

TABLE 3
Optimal temperature
of cellulase activity of DtXynA.

| ° C. | ΔOD_{414 nm} |
|---|---|
| 37 | 0.0315 |
| 45 | 0.0915 |
| 50 | 0.251 |
| 60 | 0.895 |
| 70 | 1.0219 |
| 80 | 1.1841 |
| 95 | 1.3019 |

### Example 4

### Thermostability of Cellulase Activity of DtXynA

This example shows that the cellulase activity of DtXynA is stable even after prolonged incubation at temperatures ranging from 70° C. to 95° C.

Methods:

Recombinant Enzyme Production:

cells expressing recombinant DtXynA were produced as described above in Example 1.

Thermostability Assay:

cells were centrifuged at 8000×g for 5 minutes. The supernatant was discarded, and cells were re-suspended at OD₆₀₀ₙₘ=12.5 in a 0.1 M sodium phosphate buffer at pH 6.0. Cells were incubated for 1 hour at 70° C. to precipitate non-thermostable proteins, thereby partially purifying the enzyme. Non-thermostable proteins and other cellular debris were eliminated by centrifugation at 16000×g for 20 minutes.

Aliquots of the supernatant were further inactivated for 30 minutes at 70° C. or 80° C. An aliquot was also not inactivated to serve as a control. After the incubation, 200 μl of 10% Avicel® (Sigma) was added to each aliquot, and the reaction was allowed to proceed for 5 hours at 95° C. After the 5 hour incubation, enzymatic activity was detected with a DNS colorimetric assay for reducing sugars as described in Miller (1959), supra.

Results:

As shown in Table 4, DtXynA retained its thermostable cellulase activity whether further incubated at 70° C. or 80° C.

TABLE 4
Thermostability of cellulase activity of DtXynA.

| | After 30′ | After 30′ |
|---|---|---|
| Without Further | Incubation at | Incubation at |
| Incubation | 70° C. | 80° C. |
| 100 | 129 | 108 |

### Example 5

### Xylanase Activity of DtXynA Under Alkaline Conditions

This example shows that the xylanase activity of DtXynA is present at pH 12.

Methods:

Recombinant Enzyme Production:

cells expressing recombinant DtXynA were produced as described above in Example 1.

Xylanase Assay:

cells were centrifuged at 8000×g for 5 minutes. The supernatant was discarded, and cells were re-suspended at OD₆₀₀ₙₘ=200 in a 0.1 M glycine buffer at pH 12 containing 7% NH₄OH. 100 μl of re-suspended cells were incubated for 1 hour at 60° C. After the 1 hour incubation, 200 μl of a 5% w/v oat spelt xylan (Sigma) solution prepared in the glycine buffer at pH 12 was added as a substrate, and the reaction was allowed to proceed for 1 hour at 60° C. After the 1 hour incubation, enzymatic activity was detected with the DNS colorimetric assay for reducing sugars as described in Miller (1959), supra.

Results:

As shown in Table 5, DtXynA retained greater than 50% of its xylanase activity after incubation at pH 12 when compared to enzyme not incubated at pH 12.

TABLE 5
Stability of xylanase activity of DtXynA at high pH.

| | Without | With | |
|---|---|---|---|
| | Incubation | Incubation | Stability % |
| DtXynA | 3.39 | 2.649 | 78 |
| (1801-orf3) | | | |
| Negative Control | 0.184 | 0.158 | — |

### Example 6

### Cellulase Activity of DtXynA Under Alkaline Conditions

This example shows that the cellulase activity of DtXynA is present at pH 12.

Methods:

Recombinant Enzyme Production:

cells expressing recombinant DtXynA were produced as described above in Example 1.

Cellulase Assay:

cells were centrifuged at 8000×g for 5 minutes. The supernatant was discarded, and cells were re-suspended to 400 at OD₆₀₀ₙₘ in a 0.1 M glycine buffer at pH 12 containing 7% NH₄OH. 100 μl of re-suspended cells were incubated for 1 hour at 60° C. After the 1 hour incubation, the cells were exposed to one of two conditions.

In the first condition (Table 6), 100 μl of a 10% w/v Avicel® (Sigma-Aldrich) solution prepared in the glycine buffer was added as a substrate, and the reaction was allowed to proceed for 20 hours at 60° C. After the 20 hour incubation, enzymatic activity was detected with the DNS colorimetric assay for reducing sugars as described in Miller (1959), supra.

In the second condition (Table 7), 100 μl of a 4 mM p-nitrophenyl β-D-cellobioside solution prepared in the glycine buffer was added as a substrate, and the reaction was allowed to proceed for 1 hour at 60° C. After the 1 hour incubation, 100 μl of 0.2 M Na₂CO₃ was added to the cells, and the released p-nitrophenol was measured at 414 nm.

Results:

Regardless of substrate, Tables 6 and 7 show that the cellulase activity of DtXynA was unaffected by incubation at pH 12.

TABLE 6
Cellulase activity of DtXynA at high pH on Avicel ®.

| | Without | With | |
|---|---|---|---|
| | Incubation | Incubation | Stability % |
| DtXynA | 0.217 | 0.223 | 109 |
| Negative Control | 0.131 | 0.129 | — |

TABLE 7
Cellulase activity of DtXynA at high pH on pNPCel.

| | Without | With | |
|---|---|---|---|
| | Incubation | Incubation | Stability % |
| DtXynA | 1.046 | 1.144 | 114 |
| Negative Control | 0.211 | 0.19 | — |

### Example 7

### Further Characterization of DtXYNA

Methods:

Recombinant Enzyme Production:

A nucleotide sequence encoding xylanase and cellulase (e.g., DtXynA; SEQ ID NO:3) was cloned into a pET24a E. coli expression vector using restriction enzyme sites that placed the nucleotide sequence in-frame downstream of an inducible T7lac promoter. The nucleotide sequence coding for this enzyme was generated by back translating the polypeptide sequence of the enzyme (SEQ ID NO:4) using the codon preference for E. coli. Expression vectors were introduced into an E. coli expression strain, BL21 Star (DE3; Invitrogen).

Recombinant E. coli isolates containing the modified pET24a expression vector were selected on standard LB agar containing 50 ug/ml kanamycin. Isolates were grown with shaking at 37° C. for 8 hours to overnight in 20 ml of LB media containing 50 ug/ml kanamycin.

20 ml of E. coli culture was transferred to 1 L of autoinduction medium (9.57 g trypton, 4.8 g yeast extract, 2 ml of 1 M MgSO₄, 1 ml of 1000× trace metals, 20 ml of 50×5052, and 20 ml of 50×M) (1000× trace metals: 36 ml sterile water, 50 ml of 0.1 M FeCl₃ in 0.12 M HCl, 2 ml of 1 M CaCl₂, 1 ml of 1 M MnCl₂4H₂0, 1 ml of 1 M ZnSO₄7H₂0, 1 ml of 0.2 M CoCl₂6H₂0, 2 ml of 0.1 M CuCl₂H₂0, 1 ml of 0.2 M NiCl₂6H₂0, 2 ml of 0.1 M Na₂MoO₄2H₂0, and 2 ml of 0.1 M H₃BO₃) (50×5052: 25 g glycerol, 73 ml H₂0, 2.5 g glucose, and 10 g α-lactose monohydrate) (50×M: 80 ml H₂0, 17.75 g Na₂HPO₄, 17.0 g KH₂PO₄, 13.4 g NH₄Cl, and 3.55 g Na₂SO₄) with 25 ug/ml kanamycin, and grown with shaking at 28° C. overnight.

The E. coli cells were harvested out of the autoinduction medium by centrifugation at 10,000×g for 15 minutes, and the collected cells were frozen at −80° C. Cells were lysed by re-suspending the cell pellet in a buffer (50 mM Tris, pH 7.5, 50 mM NaCl, 0.1 mM EDTA) containing DNase (1 U/ml buffer) and using a French Press. Insoluble debris was removed by centrifugation at 12,000×g for 20 minutes at 4° C.

The supernatant containing total soluble protein and recombinant enzyme was transferred to a fresh 40 ml centrifuge tube and was incubated at 75° C. for 30 minutes. The xylanases remained soluble and were separated from precipitated protein by centrifugation at 12,000×g for 30 minutes, and the collected proteins were available for further characterization.

Enzyme purity was determined using SDS-PAGE and densitometry.

Xylanase Activity During Pretreatment in Aqueous Ammonia:

Xylanase enzyme preparations as described herein (Example 7) were added to birchwood substrate-based biomass to determine xylanase and/or cellulase activity during various biomass pretreatments. The pretreatment cocktail included 0.75% birchwood xylan and varying concentrations of aqueous ammonia. The pH of each treatment was between pH 11.3 and pH 12.4.

The quantity of enzyme used was determined by a dose response curve, in which various amounts of enzyme were added to an assay performed with 0.75% birchwood xylan and 50 mM Tris, pH 8.0, and incubated for 1 hour. The optimal amount of xylanase was determined to be a maximal amount that did not saturate the birchwood xylan substrate. This amount was 1.0 μg per mg of xylan for DtXynA.

The pretreatment cocktail contained 0.75% birchwood xylan, xylanase enzyme preparation and aqueous ammonia. The pretreatment cocktail was incubated at various temperatures for 1 hour prior to measuring xylanase activity using a β-xylosidase digestion and xylose detection method as described below.

The β-xylosidase digestion hydrolyzes xylose oligosaccharides released by the xylanase into xylose monomers. These xylose monomers were detected by a colorimetric assay using xylose mutarotase and β-xylose dehydrogenase using a kit that is commercially available from Megazyme Int'l Ireland Ltd.

Xylanase Activity after Pretreatment in Aqueous Ammonia:

Stability of the E. coli-produced xylanases was determined by incubating the preparations described herein (Example 7) in varying concentrations of aqueous ammonia at 60° C., followed by neutralization and subsequent analysis of xylanase and/or cellulase activity. As a control, analysis also was performed with M1 xylanase (Megazyme Int'l Ireland Ltd.) from Trichoderma viride. The same amounts of xylanase enzyme preparation were used in these assays as in those described herein (Example 7); M1 was used at 1 μg per mg of xylan (i.e., the same concentration as DtXynA).

The xylanases were incubated in the presence of birchwood xylan substrate at 60° C. in varying concentrations of aqueous ammonia. A control sample was neutralized immediately (AAPT 0 hour), while test samples were incubated in the aqueous ammonia for 1 hour. The samples were neutralized by incubating with Dowex™ Weak Acid Resin (Dow Chemical Co.; Midland, Mich.) plus additional birchwood xylan for either 0 hour (ACT 0 hour control) or for 1 hour (ACT 1 hour test samples). Xylanase activity was determined by the DNS assay as described herein (Example 7).

Cellulase Activity after Pretreatment in Aqueous Ammonia:

The stability of the E. coli-produced xylanases was determined by incubating the preparations described herein (Example 7) in varying concentrations of aqueous ammonia at 60° C., followed by neutralization and subsequent analysis of cellulase activity as described above with the following modifications.

The positive control was Trichoderma longibrachiatum endoglucanase II (EGII; Megazyme Intl Ireland Ltd.), which is known to have cellulase activity. To measure cellulase activity, 0.25% CMC was used. Both DtXynA and EGII were used at a concentration of 20 μg per mg of CMC. After neutralization, the enzyme was incubated with the CMC substrate for 4 hours at 60° C. As noted above, the increase in incubation time for the activity assay is due to the different nature of cellulases, which have slower activity compared to xylanases. Cellulase activity was determined by the DNS colorimetric assay as described above.

Results:

Table 8 shows the relative activity of the DtXynA xylanase during an aqueous ammonia pretreatment, which was calculated by setting the enzyme activity observed at 60° C. and 0.01% aqueous ammonia to 100% and comparing the activity of the same enzyme under different temperatures and ammonia concentration to the activity at 60° C. and 0.01% aqueous ammonia.

TABLE 8
DtXynA xylanase activity during pretreatment.

| Percentage of aqueous | | | |
|---|---|---|---|
| ammonia | 50° C. | 60° C. | 80° C. |
| 0.01 | 17.8 | 100.0 | 118.6 |
| 0.1 | 18.6 | 67.2 | 91.0 |
| 1 | 17.7 | 28.0 | 40.4 |
| 4 | 15.3 | 6.4 | 9.6 |
| 8 | 15.3 | 0.0 | 2.5 |

The DtXynA xylanase is therefore active during an aqueous ammonia pretreatment. During the incubation in aqueous ammonia, the DtXynA xylanase was able to hydrolyze the birchwood xylan substrate, suggesting that this enzyme may be used during the pretreatment of cellulosic biomass to convert the biomass to fermentable sugars.

Sensitivity of the DtXynA enzyme to temperature while increasing the concentration of aqueous ammonia will be important to consider when selecting an appropriate xylanase for pretreatment of cellulosic biomass.

Table 10 shows that DtXynA retains xylanase activity after incubation in ammonia-based pretreatment conditions with a biomass substrate present in contrast to the control enzyme, M1 (Table 9).

TABLE 9
M1 xylanase activity (mM xylose equivalents) with aqueous ammonia
pretreatment (AAPT) followed by neutralization and an hour activity
incubation (AI).

| Percentage | | | | |
|---|---|---|---|---|
| of aqueous | AAPT: 0 hr, | AAPT: 0 hr, | AAPT: 1 hr, | AAPT: 1 hr, |
| ammonia | AI: 0 hr | AI: 1 hr | AI: 0 hr | AI: 1 hr |
| 0.01 | 37.8 | 100.0 | 18.2 | 19.3 |
| 0.1 | 72.7 | 129.7 | 10.8 | 9.6 |
| 1 | 56.1 | 107.1 | 7.3 | 6.2 |
| 4 | 51.7 | 97.1 | 5.4 | 5.3 |
| 8 | 37.8 | 100.0 | 18.2 | 19.3 |

Expressed as percentage, where 100% = activity at 0.01% AA,
AAPT: 0 hr,
Act: 1 hr.

TABLE 10
DtXynA xylanase activity (mM xylose equivalents) with aqueous
ammonia pretreatment (AAPT) followed by neutralization and an
hour activity incubation (AI).

| Percentage | | | | |
|---|---|---|---|---|
| of aqueous | AAPT: 0 hr, | AAPT: 0 hr, | AAPT: 1 hr, | AAPT: 1 hr, |
| ammonia | AI: 0 hr | AI: 1 hr | AI: 0 hr | AI: 1 hr |
| 0.01 | 23.2 | 100.0 | 48.6 | 143.7 |
| 0.1 | 14.1 | 27.0 | 29.9 | 88.5 |
| 1 | 8.8 | 9.9 | 20.8 | 43.8 |
| 4 | 8.3 | 10.5 | 8.2 | 20.4 |
| 8 | 23.2 | 100.0 | 48.6 | 143.7 |

Expressed as percentage, where 100% = activity at 0.01% AA,
AAPT: 0 hr,
Act: 1 hr.

The DtXynA xylanase therefore can be used during ammonia-based pretreatments and continue to function after pretreatment of biomass during the cellulosic conversion of biomass to fermentable sugars process.

The M1 enzyme (control) showed little to no tolerance to exposure to an ammonia-based pretreatment, while DtXynA showed tolerance to the ammonia-based pretreatment. The DtXynA enzyme may be an enzyme of choice when considering the development of a cellulosic biomass conversion process that includes the use of enzymes capable of breaking down biomass.

Tables 11 and 12 shows that DtXynA has cellulase activity and that this activity survives incubation in ammonia-based pretreatment conditions with a cellulase substrate present.

TABLE 11
EGII cellulase activity (mM glucose equivalents) with AAPT
followed by neutralization and an hour AI.

| Percentage | | | | |
|---|---|---|---|---|
| of aqueous | AAPT: 0 hr, | AAPT: 0 hr, | AAPT: 1 hr, | AAPT: 1 hr, |
| ammonia | AI: 0 hr | AI: 4 hr | AI: 0 hr | AI: 4 hr |
| 0.01 | 68.1 | 100.0 | 68.9 | 100.2 |
| 0.1 | 18.4 | 54.1 | 6.0 | 10.5 |
| 1 | 17.1 | 75.9 | 6.1 | 6.3 |
| 4 | 17.2 | 72.9 | 6.1 | 6.4 |
| 8 | 68.1 | 100.0 | 68.9 | 100.2 |

Expressed as percentage, where 100% = activity at 0.01% AA,
AAPT: 0 hr,
Act: 4 hr.

TABLE 12
DtXynA cellulase activity (mM glucose equivalents) with AAPT
followed by neutralization and an hour AI.

| Percentage | | | | |
|---|---|---|---|---|
| of aqueous | AAPT: 0 hr, | AAPT: 0 hr, | AAPT: 1 hr, | AAPT: 1 hr, |
| ammonia | AI: 0 hr | AI: 4 hr | AI: 0 hr | AI: 4 hr |
| 0.01 | 31.1 | 100.0 | 33.4 | 136.1 |
| 0.1 | 31.1 | 37.3 | 32.4 | 35.5 |
| 1 | 31.5 | 58.8 | 32.2 | 42.8 |
| 4 | 29.4 | 53.4 | 29.2 | 37.1 |
| 8 | 31.1 | 100.0 | 33.4 | 136.1 |

Expressed as percentage, where 100% = activity at 0.01% AA,
AAPT: 0 hr,
Act: 4 hr.

DtXynA therefore can be used as a dual activity enzyme, for both xylanase and cellulase activity, during an ammonia-based pretreatment and continue to function after pretreatment during the saccharification process, acting on both xylan and cellulose substrates. The EGII enzyme (control; Table 11) showed little to no tolerance to exposure to an ammonia-based pretreatment.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method of processing lignocellulosic material, comprising contacting the lignocellulosic material with an alkaline composition at a pH of at least about 10 and an enzyme comprising the amino acid sequence of SEQ ID NO:4, whereby the lignocellulosic material is processed.

2. The method of claim 1, wherein the lignocellulosic material is provided by a plant, plant part and/or plant cell transformed with a nucleotide sequence encoding the amino acid sequence of SEQ ID NO:4, thereby providing the enzyme comprising the amino acid sequence of SEQ ID NO:4 in the lignocellulosic material.

3. The method of claim 2, wherein the lignocellulosic material further comprises a plant, plant part or plant cell not transformed with an enzyme comprising the amino acid sequence of SEQ ID NO:4.

4. The method of claim 1 wherein the contacting is at a pH of about 10 to about 12.

5. The method of claim 1 wherein the contacting is at a temperature of about 95° C.

6. The method of claim 1 wherein the enzyme is present in an amount from about 0.01 units to about 1000 units.

7. The method of claim 1 wherein the enzyme is present in an amount of about 500 units.

8. The method of claim 1 where the contacting is for about 12 hours to about 24 hours.

9. The method of claim 1 wherein the contacting is for about 6 hours.

10. The method of claim 1 wherein the pH is reduced over time to a pH from about 6 to about 8.

11. The method of claim 10, further comprising contacting the lignocellulosic material with at least one additional enzyme selected from the group consisting of a cellulase, hemicellulase, ligninase, pectinase, protease, and any combination thereof.

12. The method of claim 11, wherein the cellulase is provided by the enzyme comprising the amino acid sequence of SEQ ID NO:4.

13. The method of claim 9 wherein the method further comprises contacting the lignocellulosic material with at least one enzyme selected from the group consisting of an amylase, catalase, cutinase, glucanase, glucoamylase, glucose isomerase, lipase, phytase, pullulanase, xylose isomerase, and any combination thereof.

14. The method of claim 9 wherein the method further comprises contacting the lignocellulosic material with an ethanologenic bacteria or yeast or a combination thereof.

15. The method of claim 1 further comprising pretreating the lignocellulosic material with a biological pretreatment, a chemical pretreatment, a physical pretreatment, or any combination thereof.

16. The method of claim 15, wherein the biological pretreatment comprises pretreating the lignocellulosic material with a lignin-solubilizing microorganism.

17. The method of claim 15, wherein the chemical pretreatment comprises pretreating the lignocellulosic material with a dilute acid treatment, an organic solvent treatment, an ozone treatment, an sulfur dioxide treatment, a carbon dioxide treatment, a pH-controlled hydrothermolysis, or any combination thereof.

18. The method of claim 15, wherein the physical pretreatment comprises pretreating the lignocellulosic material with hydrothermolysis, irradiation, milling, steaming/steam explosion, sonication, or any combination thereof.

19. The method of claim 1 wherein the method further comprises contacting the lignocellulosic material with at least one agent selected from the group consisting of a chlorine, detergent, hypochlorite, hydrogen peroxide, oxalic acid, peracid, pH-regulating agent, trisodium phosphate, sodium chlorite, sodium nitrate, surfactant, urea, water, and any combination thereof.
